(19) United States
(12) Patent Application Publication
(10) Pub. No.: US 2024/0390362 A1
(43) Pub. Date: Nov. 28, 2024

(54) METHODS OF TREATING ERYTHROPOIETIC PROTOPORPHYRIA, X-LINKED PROTOPORPHYRIA, OR CONGENITAL ERYTHROPOIETIC PORPHYRIA WITH A SOLID FORM OF BITOPERTIN

(71) Applicant: Disc Medicine, Inc., Watertown, MA (US)

(72) Inventors: Brian Richard MacDonald, Watertown, MA (US); Maria Gabriela Beconi, Watertown, MA (US); Vu Hong, Watertown, MA (US)

(21) Appl. No.: 18/562,510

(22) PCT Filed: May 26, 2022
(86) PCT No.: PCT/US2022/031081

§ 371 Date: Nov. 20, 2023

Related U.S. Application Data
(60) Provisional application No. 63/193,898, filed on May 27, 2021.

(51) Int. Cl. A61K 31/496; A61K 45/06; A61P 3/00; A61P 7/00

(52) U.S. Cl. CPC A61K 31/496; A61K 45/06; A61P 3/00; A61P 7/00

(57) ABSTRACT

The present embodiments are directed to methods of using a crystalline form (such as form A, B, or C, or a methylparaben cocrystal form) or an amorphous form of Bitopertin, or pharmaceutical compositions thereof, for preventing or treating erythropoietic protoporphyria (EPP), X-linked protoporphyria (XLPP), and/or congenital erythropoietic porphyria (CEP), and related syndromes thereof.

### RELATED APPLICATIONS

This application claims the benefit of priority from U.S. Provisional Patent Application No. 63/193,898, filed May 27, 2021. The specification of the foregoing application is incorporated herein by reference in its entirety.

### FIELD

Embodiments disclosed herein are directed to methods and uses to prevent or treat erythropoietic protoporphyria (EPP), X-linked protoporphyria (XLPP), or congenital erythropoietic porphyria (CEP) with a solid form (such as a crystalline form or an amorphous form) of Bitopertin.

### BACKGROUND

Erythropoietic protoporphyria (EPP) is prevalent globally and affects about 5,000-10,000 individuals worldwide (Michaels et al. 2010). EPP is considered the most common form of porphyria in children. Erythropoietic protoporphyria is a form of porphyria, which varies in severity and can be very painful. It arises from a deficiency in the enzyme ferrochelatase, leading to abnormally high levels of protoporphyrin IX in red blood cells (erythrocytes), plasma, skin, and liver. Erythropoietic protoporphyria (EPP) is due to an inherited or acquired deficiency in the activity of the enzyme ferrochelatase. X-linked protoporphyria (XLPP) is due to an inherited increase in the activity of delta-aminolevulinic acid synthase-2 (ALAS2). Enzymes that cause both EPP and XLPP are in the heme biosynthetic pathway. EPP and XLPP are nearly identical clinically. Congenital erythropoietic porphyria (CEP), also known as Gunther disease, caused by mutations in the gene for uroporphyrinogen synthase resulting in reduced activity of this enzyme and accumulation of the upstream metabolite coproporphyrin I. Current treatments for erythropoietic protoporphyria (EPP), X-linked protoporphyria (XLPP), or congenital erythropoietic porphyria (CEP) are limited.

Thus, there is a need for new methods and compositions for treating and/or preventing erythropoietic protoporphyria, X-linked protoporphyria, and congenital erythropoietic porphyria. The methods and uses of a solid form of Bitopertin, as described herein, fulfill these needs as well as others.

### SUMMARY OF THE APPLICATION

The present application provides a method of treating erythropoietic protoporphyria (EPP), X-linked protoporphyria (XLPP), or congenital erythropoietic porphyria (CEP) in a subject, the method comprising administering to the subject a crystalline form or an amorphous form of Bitopertin or a pharmaceutical composition comprising a crystalline form or an amorphous form of Bitopertin.

Bitopertin has an IUPAC name of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone, and has the following chemical structure:

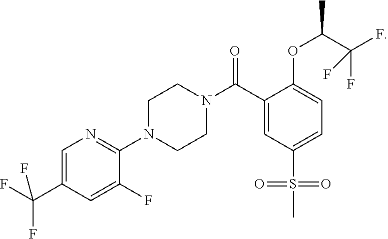 [SMILES: C[C@H](Oc1ccc(S(C)(=O)=O)cc1C(=O)N1CCN(c2ncc(C(F)(F)F)cc2F)CC1)C(F)(F)F]

Bitopertin has a CAS Registry Number® of 845614-11-1. Other names for Bitopertin include, but are not limited to: [4-(3-fluoro-5-trifluoromethyl-pyridin-2-yl)-piperazin-1-yl]-[5-methanesulfonyl-2-((S)-2,2,2-trifluoro-1-methyl-ethoxy)-phenyl]-methanone; methanone, [4-[3-fluoro-5-(trifluoromethyl)-2-pyridinyl]-1-piperazinyl][5-(methylsulfonyl)-2-[(1S)-2,2,2-trifluoro-1-methylethoxy]phenyl]-; piperazine, 1-[3-fluoro-5-(trifluoromethyl)-2-pyridinyl]-4-[5-(methylsulfonyl)-2-[(1S)-2,2,2-trifluoro-1-methylethoxy]benzoyl]-; [4-[3-fluoro-5-(trifluoromethyl)-2-pyridinyl]-1-piperazinyl][5-(methylsulfonyl)-2-[(1S)-2,2,2-trifluoro-1-methylethoxy]phenyl]methanone; (S)-[4-(3-fluoro-5-trifluoromethylpyridin-2-yl) piperazin-1-yl][5-(methanesulfonyl)-2-(2,2,2-trifluoro-1-methylethoxy)phenyl]methanone; [4-[3-fluoro-5-(trifluoromethyl) pyridin-2-yl]piperazin-1-yl]-[5-methylsulfonyl-2-[(2S)-1,1,1-trifluoropropan-2-yl]oxyphenyl]methanone. In the present application, the term “Bitopertin” is used interchangeably with its other names and/or its chemical structure. The solid forms of Bitopertin suitable for use in the present application include, but are not limited to, the solid forms described in published PCT application WO 2008/080821. The entire content of WO 2008/080821 is hereby incorporated by reference.

The present application further provides a method of preventing, treating, or reducing the progression rate and/or severity of one or more complications of EPP, XLPP, or CEP in a subject, the method comprising administering to the subject a crystalline form or an amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone, or a pharmaceutical composition comprising such a crystalline form or amorphous form. In certain embodiments, the one or more complications of EPP, XLPP, or CEP is selected from the group consisting of: acute photosensitivity, cutaneous photosensitivity, edema, erythema, anemia, hypochromic anemia, hemolytic anemia, hemolysis, mild hemolysis, severe hemolysis, chronic hemolysis, hypersplenism, palmar keratoderma, bullae, lesions, scarring, deformities, loss of fingernails, loss of digits, cholestasis, cytolysis, gallstones, cholestatic liver failure, cholelithiasis, mild liver disease, deteriorating liver disease, terminal phase liver disease, erythrodontia, hypercellular bone marrow, myelodysplasia, thrombocytopenia, hydrops fetalis and/or death in utero. In certain such embodiments, the acute photosensitivity is due to sun exposure.

The present application further provides a method for use in preventing or treating EPP, XLPP, or CEP in a subject, wherein the use comprises administering to the subject a crystalline form or an amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone, or a pharmaceutical composition comprising such a crystalline form or amorphous form.

The present application further provides a method for use in the manufacture of a medicament for the treatment of EPP, XLPP, or CEP in a subject, the use comprising administering to the subject a crystalline form or an amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone, or a pharmaceutical composition comprising such a crystalline form or amorphous form.

The present application further provides a method for use in the manufacture of a medicament for inhibiting protoporphyrin IX (PPIX) synthesis in vivo, the use comprising administering to a subject a crystalline form or an amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone, or a pharmaceutical composition comprising such a crystalline form or amorphous form.

In certain embodiments, the subject has EPP. In other embodiments, the subject has XLPP. In yet other embodiments, the subject has CEP.

In certain embodiments, the method increases pain free light exposure in the subject. In other embodiments, the method decreases light sensitivity in the subject.

The present application further provides a method of inhibiting PPIX synthesis in vivo, comprising administering to a subject a crystalline form or an amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone, or a pharmaceutical composition comprising such a crystalline form or amorphous form.

The present application further provides a method of inhibiting zinc protoporphyrin IX (ZPPIX) synthesis in vivo, comprising administering to a subject a crystalline form or an amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone, or a pharmaceutical composition comprising such a crystalline form or amorphous form.

The present application further provides a method of inhibiting uroporphyrin I and/or coproporphyrin I synthesis in vivo, comprising administering to a subject a crystalline form or an amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone, or a pharmaceutical composition comprising such a crystalline form or amorphous form.

The present application further provides a method of inhibiting 5-aminolevulinic acid (5-ALA) synthesis in vivo, comprising administering to a subject a crystalline form or an amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone, or a pharmaceutical composition comprising such a crystalline form or amorphous form.

In certain embodiments, the accumulation of one or more heme intermediates is inhibited, and wherein the one or more heme intermediates are selected from the group consisting of PPIX, ZPPIX, uroporphyrin I, coproporphyrin I, and/or 5-ALA. In certain such embodiments, the accumulation of the one or more heme intermediates is inhibited in a dose dependent manner.

In certain embodiments, the crystalline form or amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein demonstrates an EC50 of less than 500 nM. In certain embodiments, the crystalline form or amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone demonstrates an EC50 of less than 100 nM.

In certain embodiments, at least 50% cell viability is maintained. In certain embodiments, at least 90% cell viability is maintained.

In certain embodiments, the subject has PPIX levels that are at least 10%, 20%, 30%, 40%, or 50% more than PPIX levels in a healthy subject prior to administration of the crystalline form or amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

In certain embodiments, the subject has ZPPIX levels that are at least 10%, 20%, 30%, 40%, or 50% more than ZPPIX levels in a healthy subject prior to administration of crystalline form or amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

In certain embodiments, the subject has increased proportion of ZPPIX to free-protoporphyrin IX (ZPPIX/PPIX ratio) as compared to those with EPP.

In certain embodiments, the subject has uroporphyrin I and/or coproporphyrin I levels that are at least 10%, 20%, 30%, 40%, or 50% more than uroporphyrin I and/or coproporphyrin I levels in a healthy subject prior to administration of the crystalline form or amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

In certain embodiments, the subject has 5-ALA levels that are at least 10%, 20%, 30%, 40%, or 50% more than 5-ALA levels in a healthy subject prior to administration of the crystalline form or amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone. In certain embodiments, the subject's PPIX levels decrease while the patient's heme levels are substantially maintained. In certain embodiments, the patient's PPIX levels decrease by at least 50% (e.g., 50%, 55%, 60%, 65%, 70%, 75%, 80%, 85%, 90%, 95%, or at least 100%) and the patient's heme levels decrease no more than 10% (e.g., 10%, 15%, 20%, 25%, and 30%). In certain embodiments, the patient's PPIX levels decrease by at least 85% and the patient's heme levels decrease no more than 15%. In certain embodiments, heme levels decrease no more than 10% (e.g., 10%, 15%, 20%, 25%, and 30%). In certain embodiments, the dosage of the pharmaceutical composition does not cause a substantial reduction in heme levels.

In certain embodiments, the subject has increased free-protoporphyrin IX levels in erythrocytes. In certain embodiments, the method decreases free-protoporphyrin IX levels in the subject. In certain such embodiments, the method decreases free-protoporphyrin IX levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In certain embodiments, the subject has increased protoporphyrin IX levels in the stool. In certain embodiments, the method decreases protoporphyrin IX levels in the stool of the subject. In certain such embodiments, the method decreases protoporphyrin IX levels in the stool of the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%).

In certain embodiments, the subject's plasma porphyrin fluoresces at a peak of 634 nm when illuminated with blue light (e.g., 400-420 nm light). In certain embodiments, the subject's plasma porphyrin fluoresces at a peak between 626 nm and 634 nm when illuminated with blue light (e.g., 400-420 nm light). In certain embodiments, the subject's skin porphyrin fluoresces at a peak of 632 nm when illuminated with blue light (e.g., 400-420 nm light). In certain embodiments, the subject's skin porphyrin fluoresces at a peak between 626 nm and 634 nm when illuminated with blue light (e.g., 400-420 nm light).

In certain embodiments, the subject has increased protoporphyrin IX levels in the skin. In certain embodiments, the method decreases protoporphyrin IX levels in the skin of the subject. In certain such embodiments, the method decreases protoporphyrin IX levels in the skin of the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In certain embodiments, the subject has greater than 0.2 FluoDerm Units (FDU) of protoporphyrin IX levels in the skin. In certain embodiments, the subject has greater than 1.0 FDU of protoporphyrin IX levels in the skin. In certain embodiments, the subject has between 1.0 FDU and 2.5 FDU of protoporphyrin IX levels in the skin. In certain embodiments, the subject has greater than 2.5 FDU of protoporphyrin IX levels in the skin. In certain embodiments, the method decreases protoporphyrin IX levels in the skin of the subject to less than 0.5 FDU. In certain embodiments, the method decreases protoporphyrin IX levels in the skin of the subject to less than 1.0 FDU. In certain embodiments, the method decreases protoporphyrin IX levels in the skin of the subject to less than 1.5 FDU. In certain embodiments, the method decreases protoporphyrin IX levels in the skin of the subject to less than 2.0 FDU. In certain embodiments, the method decreases protoporphyrin IX levels in the skin of the subject to less than 2.5 FDU.

In certain embodiments, the subject has increased protoporphyrin IX levels in the erythrocytes. In certain embodiments, the method decreases protoporphyrin IX levels in the erythrocytes of the subject. In certain such embodiments, the method decreases protoporphyrin IX levels in the erythrocytes of the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In certain embodiments, the subject has greater than 31 μmol L⁻¹ protoporphyrin IX levels in the erythrocytes. In certain embodiments, the subject has between 31 μmol L⁻¹ and 53 μmol L⁻¹ protoporphyrin IX levels in the erythrocytes. In certain embodiments, the subject has greater than 53 μmol L⁻¹ protoporphyrin IX levels in the erythrocytes. In certain embodiments, the method decreases protoporphyrin IX levels in the erythrocytes of the subject to levels less than 53 μmol L⁻¹. In certain embodiments, the method decreases protoporphyrin IX levels in the erythrocytes of the subject to levels less than 31 μmol L⁻¹. In certain embodiments, the method decreases protoporphyrin IX levels in the erythrocytes of the subject to levels less than 15 μmol L^{-1.}

In certain embodiments, the subject's ferrochelatase activity level is reduced to between 10 to 35% of the ferrocheletase activity level observed in normal subjects. In certain embodiments, the subject's ferrochelatase activity level is reduced to less than 50% of the ferrocheletase activity level observed in normal subjects.

In certain embodiments, the subject has a gain-of-function mutation in ALAS2. In certain embodiments, the subject's ALAS2 enzyme activity is increased.

In certain embodiments, the subject has increased zinc-protoporphyrin IX levels in erythrocytes. In certain embodiments, the method decreases zinc-protoporphyrin IX levels in the subject's erythrocytes. In certain such embodiments, the method decreases zinc-protoporphyrin IX levels in the subject's erythrocytes by at least 10% (e.g., 10%, 15%, 20%, 25%, 30%, 35%, 40%, 45%, 50%, 55%, 60%, 65%, 70%, 75%, 80%, 85%, 90%, 95%, or at least 100%).

In certain embodiments, the subject has decreased activity of uroporphyrinogen III synthase. In certain embodiments, the subject has increased levels of uroporphyrin I and/or coproporphyrin I. In certain embodiments, the increased levels of uroporphyrin I and/or coproporphyrin I are measured in the subject's urine or red blood cells. In certain embodiments, the increased levels of coproporphyrin I are measured in the subject's stool. In certain embodiments, the method decreases the subject's levels of uroporphyrin I and/or coproporphyrin I. In certain embodiments, the method decreases the subject's levels of uroporphyrin I. In certain such embodiments, the method decreases the subject's levels of uroporphyrin I by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In certain embodiments, the method decreases the subject's levels of coproporphyrin I. In certain such embodiments, the method decreases the subject's levels of coproporphyrin I by at least 10% (e.g., 10%, 15%, 20%, 25%, 30%, 35%, 40%, 45%, 50%, 55%, 60%, 65%, 70%, 75%, 80%, 85%, 90%, 95%, or at least 100%).

In certain embodiments, the subject has a mutation in UROS.

In certain embodiments, the subject has a gene defect in GATA-1 erythroid-specific transcription factor.

In certain embodiments, the subject has red fluorescent urine. In certain embodiments, the subject has a peak between 615 nm and 620 nm using plasma porphyrin fluorescence analysis.

In certain embodiments, the subject has liver disease associated with EPP, XLPP, or

CEP. In certain embodiments, the liver disease associated with EPP, XLPP, or CEP is cholelithiasis. In certain embodiments, the liver disease associated with EPP, XLPP, or CEP is mild liver disease. In certain embodiments, the liver disease associated with EPP, XLPP, or CEP is deteriorating liver disease. In certain embodiments, the liver disease associated with EPP, XLPP, or CEP is terminal phase liver disease.

In certain embodiments, the method further comprises administering to the subject an additional active agent and/or supportive therapy. In certain such embodiments, the additional active agent and/or supportive therapy is selected from the group consisting of: avoiding sunlight, topical sunscreens, skin protection, UVB phototherapy, Afamelanotide (Scenesse®), bortezomib, proteasome inhibitors, chemical chaperones, cholestyramine, activated charcoal, iron supplementation, liver transplantation, bone marrow transplantation, splenectomy, and blood transfusion.

The methods or uses described herein relate to several distinct crystalline forms and an amorphous form of Bitopertin

In certain embodiments of the methods or uses of this application, the solid form of Bitopertin is selected from the crystalline or amorphous forms described in published PCT application WO 2008/080821, the entire content of which is incorporated herein by reference.

In certain embodiments of the methods or uses of this application, the crystalline form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone is selected from the group consisting of three distinct crystalline forms A, B, and C of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

In certain embodiments of the methods or uses of this application, the crystalline form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone is a methylparaben cocrystal form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

In certain embodiments, the methods or uses of this application relate to an amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

In certain embodiments, the methods or uses of this application relate to a pharmaceutical composition comprising a form A, a form B, a form C, a methylparaben cocrystal form, or an amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone as an active ingredient.

In some embodiments, the solid forms of Bitopertin described herein can be distinguished by physical and chemical properties that can be characterized by infra-red spectra, X-ray powder diffraction patterns, melting behavior or glass transition temperatures. In certain embodiments, the pharmaceutical composition further comprises a pharmaceutically acceptable carrier.

In certain embodiments, the subject is a subject in need thereof.

In certain embodiments of the methods or uses described herein, the crystalline form or the amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone is administered in a therapeutically effective amount.

### BRIEF DESCRIPTION OF THE DRAWINGS

FIG. 1 shows western blot determination of ferrochelatase (FECH) protein expression levels for various K562 clones.

FIG. 2 shows flow cytometry determination of protoporphyrin IX (PPIX) levels for K562 clones.

FIG. 3 shows heme and PPIX levels of WT K562 and clone 1-9 cells as determined by LC/MS/MS

FIG. 4 shows the inhibition of PPIX formation by Bitopertin and PF-03463275 as determined by flow cytometry.

FIG. 5 shows the effects of Bitopertin and PF-03463275 on cell viability as measured by Vi-CELL XR Complete System.

FIG. 6 shows the effect of Bitopertin treatment on 5-Aminolevulinic acid (also referred to as 8-aminolevulinic acid or 5-ALA) level of clone 1-9 cells.

FIG. 7 shows the effect of Bitopertin treatment on PPIX level of clone 1-9 cells.

FIG. 8 shows the effect of Bitopertin treatment on Heme level of clone 1-9 cells.

FIG. 9 shows the relative FECH mRNA levels in human hematopoietic stem cells after transduction with lentiviral vectors expressing shRNA of FECH.

FIG. 10 shows flow cytometry determination of the effects of Bitopertin treatment on the erythroid cell antigen profile and the protoporphyrin IX (PPIX) levels in the human hematopoietic stem cells.

FIG. 11 shows that Biotopertin (100 nM) treatment reduced PPIX accumulation in CD34+ cells by 60%.

FIG. 12 shows a XRPD (Powder X-Ray Powder Diffraction) pattern of a typical lot of form A of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

FIG. 13 shows an IR (Infra-Red Spectroscopy) spectrum of a typical lot of form A of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

FIG. 14 shows a DSC (Differential Scanning Calorimetry) curve of a typical lot of form A of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

FIG. 15 shows a TGA (Thermo Gravimetric Analysis) curve of a typical lot of form A of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

FIG. 16 shows a XRPD (Powder X-Ray Diffraction) pattern of a typical lot of form B of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

FIG. 17 shows an IR (Infra-Red) spectrum of a typical lot of form B of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

FIG. 18 shows a DSC (Differential Scanning Calorimetry) curve of a typical lot of form B of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

FIG. 19 shows a TGA (Thermo Gravimetric Analysis) curve of a typical lot of form B of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

FIG. 20 shows a XRPD (Powder X-Ray Diffraction) pattern of a typical lot of form C [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

FIG. 21 shows an IR (Infra-Red) spectrum of a typical lot of form C of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

FIG. 22 shows a DSC (Differential Scanning Calorimetry) curve of a typical lot of form C of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

FIG. 23 shows a TGA (Thermo Gravimetric Analysis) curve of a typical lot of form C of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

FIG. 24 shows a XRPD (Powder X-Ray Diffraction) pattern of a typical lot of the amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

FIG. 25 shows an IR (Infra-Red) spectrum of a typical lot of the amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

FIG. 26 shows DSC (Differential Scanning Calorimetry) curves of two typical lots of the amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

FIG. 27 shows a TGA (Thermo Gravimetric Analysis) curve of a typical lot of the amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

FIG. 28 shows a DVS (Dynamic Vapor Sorption) isotherm of a typical lot of the amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

FIG. 29 shows a XRPD (Powder X-Ray Diffraction) pattern of a typical lot of the methylparaben cocrystal form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

FIG. 30 shows an IR (Infra-Red) spectrum of a typical lot of the methylparaben cocrystal form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

FIG. 31 shows a DSC (Differential Scanning Calorimetry) curve of a typical lot of the methylparaben cocrystal form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

FIG. 32 shows a TGA (Thermo Gravimetric Analysis) curve of a typical lot of the methylparaben cocrystal form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

### DETAILED DESCRIPTION OF THE APPLICATION

Unless defined otherwise, all technical and scientific terms have the same meaning as is commonly understood by one of ordinary skill in the art to which the embodiments disclosed belongs.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

As used herein, the term “amorphous form” or “amorphous” denote a material that lacks long-range order and as such, does not show sharp X-ray peaks, i.e. a Bragg diffraction peak. The XRPD pattern of an amorphous material is characterized by one or more amorphous halos.

Bragg's law describes the diffraction of crystalline material with the equation:

2d sin theta=n lambda

wherein d=perpendicular distance between pairs of adjacent planes in a crystal (d-spacing), theta=Bragg angle, lambda=wavelength and n=integer.

When Bragg's law is fulfilled, the reflected beams are in phase and interfere constructively so that Bragg diffraction peaks are observed in the X-ray diffraction pattern.

At angles of incidence other than the Bragg angle, reflected beams are out of phase and destructive interference or cancellation occurs. Amorphous material does not satisfy Bragg's law and no Bragg diffraction peaks are observed in the X-ray diffraction pattern.

“An amorphous halo” is an approximately bell-shaped diffraction maximum in the X-ray powder diffraction pattern of an amorphous substance. The FWHM of an amorphous halo is bigger than two degrees in 2-theta. “FWHM” means full width at half maximum, which is a width of a peak appearing in an XRPD pattern at its half height.

“API” is used herein as an acronym of Active Pharmaceutical Ingredient.

As used herein, the term “carrier” means a diluent, adjuvant, or excipient with which a compound is administered. Pharmaceutical carriers can be liquids, such as water and oils, including those of petroleum, animal, vegetable or synthetic origin, such as peanut oil, soybean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

A “cocrystal” is formed between a molecular or ionic API and a cocrystal former that is a solid under ambient conditions, i.e. a cocrystal is a multi-component crystalline material comprising two or more solids (at ambient conditions).

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “contacting” means bringing together of two elements in an in vitro system or an in vivo system. For example, “contacting” a GlyT1 transporter inhibitor (such as a crystalline or amorphous form of Bitopertin as described herein) with a GlyT1 transporter with an individual or patient or cell includes the administration of the compound to an individual or patient, such as a human, as well as, for example, introducing a compound into a sample containing a cellular or purified preparation containing the GlyT1 transporter.

The term “crystalline polymorph(s)” or “polymorph(s),” as used interchangeably herein, means the solid crystalline forms of a compound or complex thereof. Different polymorphs of the same compound may exhibit different physical, chemical and/or spectroscopic properties.

“DSC” is used herein as an acronym of Differential Scanning Calorimetry. In some embodiments of this application, DSC curves were recorded using a Mettler-Toledo™ differential scanning calorimeter DSC820 or DSC 821 with a FRS05 sensor. In some embodiments, system suitability tests and calibrations were carried out according to the internal standard operation procedure.

In some embodiments for the measurements of crystalline forms, approximately 2-6 mg of a sample were placed in aluminum pans, accurately weighed and hermetically closed with perforation lids. Prior to measurement, the lids were automatically pierced resulting in approx. 1.5 mm pin holes. The samples were then heated under a flow of nitrogen of about 100 mL/min using heating rates of 10 K/min.

In some embodiments for the measurements of amorphous forms, approximately 2-6 mg of a sample were placed in aluminum pans, accurately weighed and hermetically closed. The sample was then heated under a flow of nitrogen of about 100 mL/min using heating rates of 10 K/min.

“DVS” is used herein as an acronym of Dynamic Vapor Sorption. In some embodiments of this application, DVS isotherms were collected on a DVS-I (SMS Surface Measurements Systems) moisture balance system. In some embodiments, the sorption/desorption isotherms were measured stepwise in a range of 0% RH to 90% RH at 25° C. A weight change of <0.002 mg/min was chosen as criterion to switch to the next level of relative humidity (with a maximum equilibration time of six hours, if the weight criterion was not met). The data were corrected for the initial moisture content of the samples; that is, the weight after drying the sample at 0% relative humidity was taken as the zero point.

“Form A” or “crystalline form A” or “polymorphic form A” is used herein as abbreviation for the crystalline form A of Bitopertin.

“Form B” or “crystalline form B” or “polymorphic form B” “is used herein as abbreviation for the crystalline form B of Bitopertin.

“Form C” or “crystalline form C” or “polymorphic form C” is used herein as abbreviation for the crystalline form C of Bitopertin.

As used herein, the term “glycine transporter” or “GlyT” refers to membrane protein that facilitates the transport of glycine across the plasma membrane of a cell. Non-limiting examples of glycine transports include glycine transporter 1 (GlyT1) and glycine transporter 2 (GlyT2).

As used herein, the term “GlyT1” or “GlyT1 transporter” means sodium- and chloride-dependent glycine transporter 1, also known as glycine transporter 1, is a protein that in humans is encoded by the SLC6A9 gene (Kim K M, Kingsmore S F, Han H, Yang-Feng T L, Godinot N, Seldin M F, Caron M G, Giros B (June 1994). “Cloning of the human glycine transporter type 1: molecular and pharmacological characterization of novel isoform variants and chromosomal localization of the gene in the human and mouse genomes”. Mol Pharmacol. 45 (4): 608-17; Jones E M, Fernald A, Bell G I, Le Beau M M (November 1995). “Assignment of SLC6A9 to human chromosome band 1p33 by in situ hybridization”. Cytogenet Cell Genet. 71 (3): 211), which is hereby incorporated by reference in its entirety.

As used herein, the term “GlyT2” or “GlyT2 transporter” means sodium- and chloride-dependent glycine transporter 2, also known as glycine transporter 2, is a protein that in humans is encoded by the SLC6A5 gene (Morrow J A, Collie I T, Dunbar D R, Walker G B, Shahid M, Hill D R (November 1998). “Molecular cloning and functional expression of the human glycine transporter GlyT2 and chromosomal localization of the gene in the human genome”. FEBS Lett. 439 (3): 334-40), which is hereby incorporated by reference in its entirety.

As used herein, the term “GlyT1 inhibitor” means a compound that inhibits or blocks the activity of GlyT1 transporter including compounds inhibiting the activity of any isoform of GlyT1. In some embodiments, the GlyT1 inhibitor is a specific GlyT1 inhibitor, which means that the inhibitor has an inhibitor activity that is greater for GlyT1 as compared to GlyT2. In some embodiments, the inhibitor inhibits GlyT1 as compared to GlyT2 with at least, or about, 10%, 20%, 30%, 40%, 50%, 60%, 70%, 80%, 90%, 95%, 96%, 97%, 98%, 99% selectivity. In some embodiments, the GlyT1 inhibitor inhibits GlyT1 but does not inhibit or significantly inhibit the activity of GlyT2. A GlyT1 inhibitor that does not significantly inhibit the activity of GlyT2 if it inhibits the activity of GlyT2 less than 5%, 4%, 3%, 2%, or 1%. The selectivity of GlyT1 inhibitor is determined based on the known assays in the art such as the assays described in the published journal article (B. N. Atkinson, S. C. Bell, M. De Vivo, L. R. Kowalski, S. M. Lechner, V. I. Ognyanov, C.-S. Tham, C. Tsai, J. Jia, D. Ashton and M. A. Klitenick, ALX 5407: A Potent, Selective Inhibitor of the hGlyT1 Glycine Transporter, Molecular Pharmacology December 2001, 60 (6) 1414-1420), which is incorporated by its entirety. In some embodiment, the GlyT1 inhibitor is a crystalline or amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone as described herein.

As used herein, the term “GlyT2 inhibitor” means a compound that inhibits or blocks the activity of GlyT2 transporter including compounds inhibiting the activity of any isoform of GlyT2. In some embodiments, the GlyT2 inhibitor is a non-specific inhibitor, which means that it can also inhibit or block the activity of GlyT1. In some embodiments, the GlyT2 inhibitor is a specific GlyT2 inhibitor, which means that the inhibitor has an inhibitor activity that is greater for GlyT2 as compared to GlyT1. In some embodiments, the inhibitor inhibits GlyT2 as compared to GlyT1 with at least, or about, 10%, 20%, 30%, 40%, 50%, 60%, 70%, 80%, 90%, 95%, 96%, 97%, 98%, 99% selectivity. In some embodiments, the GlyT2 inhibitor inhibits GlyT2 activity but does not inhibit or significantly inhibit the activity of GlyT1. A GlyT2 inhibitor that does not significantly inhibit the activity of GlyT1 if it inhibits the activity of GlyT1 less than 5%, 4%, 3%, 2%, or 1%. The selectivity of GlyT2 inhibitor is determined based on the known assays in the art such as the assays based described in the published journal article (B. N. Atkinson, S. C. Bell, M. De Vivo, L. R. Kowalski, S. M. Lechner, V. I. Ognyanov, C.-S. Tham, C. Tsai, J. Jia, D. Ashton and M. A. Klitenick, ALX 5407: A Potent, Selective Inhibitor of the hGlyT1 Glycine Transporter, Molecular Pharmacology December 2001, 60 (6) 1414-1420), which is incorporated by its entirety.

As used herein, the term “individual” or “patient,” used interchangeably, means any animal, including mammals, such as mice, rats, other rodents, rabbits, dogs, cats, swine, cattle, sheep, horses, or primates, such as humans.

As used herein, the phrase “inhibiting activity,” such as enzymatic or transporter activity means reducing by any measurable amount the activity of an enzyme or transporter, such as the GlyT1 transporter.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

As used herein, the phrase “in situ gellable” means embracing not only liquids of low viscosity that form gels upon contact with the eye or with lacrimal fluid in the exterior of the eye, but also more viscous liquids such as semi-fluid and thixotropic gels that exhibit substantially increased viscosity or gel stiffness upon administration to the eye.

“IR” is used herein as an acronym of Infra-Red or infrared, hence “IR spectrum” means Infra-Red Spectrum. In some embodiment, the IR-spectrum of a sample was recorded as film of a Nujol suspension consisting of approx. 5 mg of sample and few Nujol between two sodium chloride plates, with an FT-IR spectrometer in transmittance. The Spectrometer was a Nicolet™ 20SXB or equivalent (resolution: 2 cm^{-1, 32} or more coadded scans, MCT detector).

As used herein, the term “mammal” means a rodent (i.e., a mouse, a rat, or a guinea pig), a monkey, a cat, a dog, a cow, a horse, a pig, or a human. In some embodiments, the mammal is a human.

“Methylparaben cocrystal form” is used herein as abbreviation for the methylparaben cocrystal form of Bitopertin.

As used herein, the phrase “ophthalmically acceptable” means having no persistent detrimental effect on the treated eye or the functioning thereof, or on the general health of the subject being treated. However, it will be recognized that transient effects such as minor irritation or a “stinging” sensation are common with topical ophthalmic administration of drugs and the existence of such transient effects is not inconsistent with the composition, formulation, or ingredient (e.g., excipient) in question being “ophthalmically acceptable” as herein defined.

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

As used herein, the term “purified” means that when isolated, the isolate contains at least 90%, at least 95%, at least 98%, or at least 99% of a compound described herein by weight of the isolate.

As used herein, the phrase “solubilizing agent” means agents that result in formation of a micellar solution or a true solution of the drug.

As used herein, the term “solution/suspension” means a liquid composition wherein a first portion of the active agent is present in solution and a second portion of the active agent is present in particulate form, in suspension in a liquid matrix.

As used herein, the phrase “substantially isolated” means a compound that is at least partially or substantially separated from the environment in which it is formed or detected.

“TGA” is used herein as an acronym of Thermo Gravimetric Analysis. In some embodiments, the TGA curves were measured on a Mettler-Toledo™ thermogravimetric analyzer (TGA850 or TGA851). In certain embodiments, system suitability tests and calibrations were carried out according to the internal standard operation procedure.

For the thermogravimetric analyses, approx. 5 to 10 mg of sample were placed in aluminum pans, accurately weighed and hermetically closed with perforation lids. Prior to measurement, the lids were automatically pierced resulting in approx. 1.5 mm pin holes. The samples were then heated under a flow of nitrogen of about 50 mL/min using a heating rate of 5 K/min.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size and sex of the subject. Optimal amounts can also be determined based on monitoring of the subject's response to treatment.

As used herein, the terms “treat,” “treated,” or “treating” mean both therapeutic treatment and prophylactic measures wherein the object is to slow down (lessen) an undesired physiological condition, disorder or disease, or obtain beneficial or desired clinical results. Beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Treatment also includes prolonging survival as compared to expected survival if not receiving treatment. Thus, “treatment of erythropoietic protoporphyria” or “treating erythropoietic protoporphyria” means an activity that alleviates or ameliorates any of the primary phenomena or secondary symptoms associated with the erythropoietic protoporphyria or other condition described herein.

“XRPD” is used herein as an acronym of X-Ray Powder Diffraction. In some embodiments, X-ray diffraction patterns were recorded at ambient conditions in transmission geometry with a STOE STADI P diffractometer (Cu Ka radiation, primary monochromator, position sensitive detector, angular range 3 to 42 2Theta (deg), approximately 60 minutes total measurement time). The samples were prepared and analyzed without further processing (e.g. grinding or sieving) of the substance.

Alternatively, X-ray diffraction patterns were recorded in transmission geometry with a STOE STADIP diffractometer with CuKa radiation (1.54 A) and a position sensitive detector. The samples (approximately 50 mg) were prepared between thin polymer (or aluminum) films and analyzed without further processing (e.g. grinding or sieving) of the substance.

X-ray diffraction patterns were also measured on a Scintag X1 powder X-ray diffractometer equipped with a sealed copper Ka 1 radiation source. The samples were scanned from 2 to 36 2Theta (deg) at a rate of 1 degree 2Theta per minute with incident beam slit widths of 2 and 4 mm and diffracted beam slit widths of 0.3 and 0.2 mm.

For single crystal structure analysis a single crystal was mounted in a loop on a goniometer and measured at ambient conditions. Alternatively, the crystal was cooled in a nitrogen stream during measurement. Data were collected on a STOE Imaging Plate Diffraction System (IPDS) from STOE (Darmstadt). In this case Mo-radiation of 0.71 A wavelength was used for data collection. Data was processed with STOE IPDS-software. The crystal structure was solved and refined with standard crystallographic software. In this case the program ShelXTL from Bruker AXS (Karlsruhe) was used.

Alternatively, for synchrotron radiation was used for data collection. A single crystal was mounted in a loop and cooled to approximately 100 K in a nitrogen stream. Data was collected at the Swiss Light Source beamline XIOSA using a MAR CCD225 detector with synchrotron radiation and data processed with the program XDS. The crystal structure was solved and refined with standard crystallographic software. In this case the program ShelXTL from Bruker AXS (Karlsruhe) was used. The crystal structure was solved and refined with ShelXTL (Bruker AXS, Karlsruhe).

The term “approximately” used in the context of describing an XRPD pattern means that there is an uncertainty in the measurements of the degrees 2Theta of ±0.2 (expressed in degrees 2Theta).

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

In some embodiments, the methods and uses disclosed herein relate to a GlyT1 inhibitor, such as Bitopertin or a solid form thereof. In some embodiments, the methods and uses disclosed herein relate to a crystalline form or an amorphous form of Bitopertin, including the solid forms described in published PCT application WO 2008/080821, the entire content of which is hereby incorporated by reference.

Bitopertin can be isolated, depending upon the method of preparation, as crystalline form A, B, or C, or as a methylparaben cocrystal form, or as an amorphous form. Forms A, B and C can be isolated from several different crystallization methods as described herein. The amorphous form can be obtained by lyophilization or fast concentration of a Bitopertin solution as described herein. The methylparaben cocrystal form can be obtained by digestion or re-crystallization of form A, B, C or amorphous form and methylparaben as described herein.

In some embodiments, the methods and uses disclosed herein relate to form A. In some embodiments, form A can be prepared by a method comprising the step of:
recrystallization of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone after seeding; or
recrystallization of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone and spontaneous crystallization below about 40° C., without seeding.

In certain embodiments, form A can be obtained by recrystallization of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone in ethanol at certain temperature and concentration after seeding with subsequent crystallization during cooling. In some embodiments, form A can be obtained by recrystallization of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone in ethanol and spontaneous crystallization below 40° C., without seeding, with subsequent precipitation during cooling. In certain embodiments, the formation of form A is not limited to ethanol, ethanol/water, methanol, methanol/water, toluene, 2-propanole, dioxane/water and dioxane. Certain aspects of the methods of preparation and in particular, the preparation of seeding crystals are further described in the examples of this application.

In some embodiments, form A is a solvent-free form as no significant weight loss is observed in the TGA curve prior to decomposition.

In some embodiments, form A can be characterized by at least three peaks selected from the following X-ray diffraction peaks obtained with a CuKa radiation expressed in degrees 2Theta at approximately: 13.1, 14.3, 15.4, 16.2, 17.1, 17.2, 17.6, 18.0, 19.8, 20.1, 20.4, 21.0, 22.6, 24.3.

In some embodiments, form A can be characterized by at least five peaks selected from the following X-ray diffraction peaks obtained with a CuKa radiation expressed in degrees 2Theta at approximately: 13.1, 14.3, 15.4, 16.2, 17.1, 17.2, 17.6, 18.0, 19.8, 20.1, 20.4, 21.0, 22.6, 24.3.

In some embodiments, form A can be characterized by at least seven peaks selected from the following X-ray diffraction peaks obtained with a CuKa radiation expressed in degrees 2Theta at approximately: 13.1, 14.3, 15.4, 16.2, 17.1, 17.2, 17.6, 18.0, 19.8, 20.1, 20.4, 21.0, 22.6, 24.3.

In some embodiments, form A can also be characterized by the following X-ray diffraction peaks obtained with a CuKa radiation expressed in degrees 2Theta at approximately: 13.1, 14.3, 15.4, 16.2, 17.1, 17.2, 17.6, 18.0, 19.8, 20.1, 20.4, 21.0, 22.6 and 24.3.

The term “approximately” means in this context that there is an uncertainty in the measurements of the degrees 2Theta of +0.2 (expressed in degrees 2Theta).

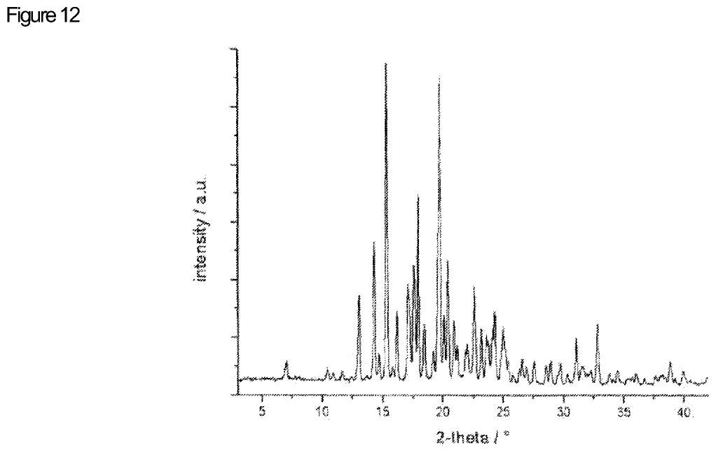

In some embodiments, form A can also be characterized by the X-ray diffraction pattern as substantially shown on FIG. 12.

In some embodiments, form A can be characterized by an infrared spectrum having sharp bands at 3032, 1645, 1623, 1600, 1581, 1501, 1342, 1331, 1314, 1291, 1266, 1245, 1154, 1130, 1088, 1054, 1012, 976, 951, 922, 889, 824, 787, 758, 739, 714 and 636 cm⁻¹ (±3 cm⁻¹).

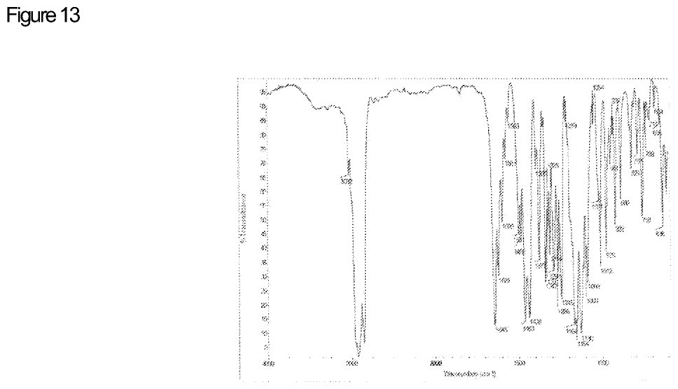

In some embodiments, form A can be characterized by the infrared spectrum as substantially shown on FIG. 13.

In some embodiments, form A can be characterized by a melting point with onset temperature (DSC) in the range of about 138° C. to 144° C.

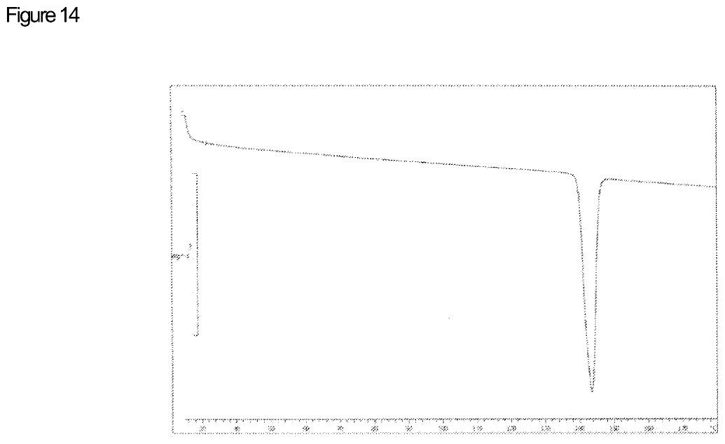

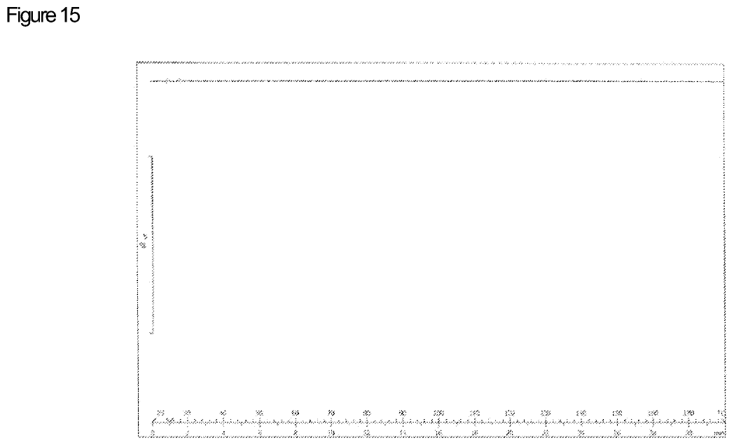

In some embodiments, certain characteristics of form A are shown in FIGS. 12 to 15.

A single crystal structure analysis of form A was conducted. Table 1 lists certain aspects of the crystal structure data. The experimental XRPD pattern collected with the form A corresponds to the theoretical pattern calculated from crystal structure data. In the single crystal structure of form A, the piperazine ring shows chair conformation with the pyridine substituent standing in equatorial position.

TABLE 1
Crystal structure data for form A crystal

| Name | Form A |
|---|---|
| Empirical Formula | C₂₁H₂₀F₇N₃O₄S |
| Formula Weight | 543.46 g · mol⁻¹ |
| Temperature | 88K |
| Space Group | P2(1)2(1)2 |
| Unit Cell Dimensions | a = 45.050(9) A |
| | alpha = 90 deg. |
| | B = 8.3500(17) A |
| | beta = 90 deg. |
| | C = 12.380(3) A |
| | gamma = 90 deg. |
| Cell Volume | 4657.0(16) A³ |
| Molecules in Unit Cell | 8 |
| Calculated Density | 1.550 g/cm³ |

In certain embodiments of the methods or uses described herein, the compound [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone comprises at least 70% of a crystalline polymorph of form A as described herein. In certain embodiments, [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone comprises at least 90% of a crystalline polymorph of form A as described herein. In certain embodiments, [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone comprises at least 96% of a crystalline polymorph of form A as described herein. In certain embodiments, [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone comprises at least 99% of a crystalline polymorph of form A as described herein.

In some embodiments, the methods and uses disclosed herein relate to form B. In certain embodiments of the application, form B can be prepared by a method comprising the step of:
seeding of a solution of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone; or
crystallization of a solution of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone without seeding; or
recrystallization of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone in one or more solvents and seeding with form B.

In certain embodiments, form B can be obtained by seeding of an ethanol solution and subsequent cooling. In certain embodiments, form B can be obtained without seeding of an ethanol solution and subsequent cooling. In certain embodiments, form B can be prepared by re-crystallization of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone in several solvents and seeding with form B.

In certain embodiments, form B is a solvent-free form as no significant weight loss is observed in the TGA curve prior to decomposition.

Certain aspects of the methods of preparation of form B, and in particular, the preparation of seeding crystals are further described in the examples of this application.

In certain embodiments, form B can be characterized by at least three peaks selected from the following X-ray diffraction peaks obtained with a CuKa radiation expressed in degrees 2Theta at approximately: 11.4, 15.4, 16.2, 16.2, 16.4, 17.8, 18.3, 19.2, 20.1, 21.0, 22.0, 22.5, 26.4.

In certain embodiments, form B can be characterized by at least five peaks selected from the following X-ray diffraction peaks obtained with a CuKa radiation expressed in degrees 2Theta at approximately: 11.4, 15.4, 16.2, 16.2, 16.4, 17.8, 18.3, 19.2, 20.1, 21.0, 22.0, 22.5, 26.4.

In certain embodiments, form B can be characterized by at least seven peaks selected from the following X-ray diffraction peaks obtained with a CuKa radiation expressed in degrees 2Theta at approximately: 11.4, 15.4, 16.2, 16.2, 16.4, 17.8, 18.3, 19.2, 20.1, 21.0, 22.0, 22.5, 26.4.

In certain embodiments, form B can also be characterized by the following X-ray diffraction peaks obtained with a CuKa radiation expressed in degrees 2Theta at approximately: 11.4, 15.4, 16.2, 16.2, 16.4, 17.8, 18.3, 19.2, 20.1, 21.0, 22.0, 22.5 and 26.4.

The term “approximately” means in this context that there is an uncertainty in the measurements of the degrees 2Theta of ±0.2 (expressed in degrees 2Theta).

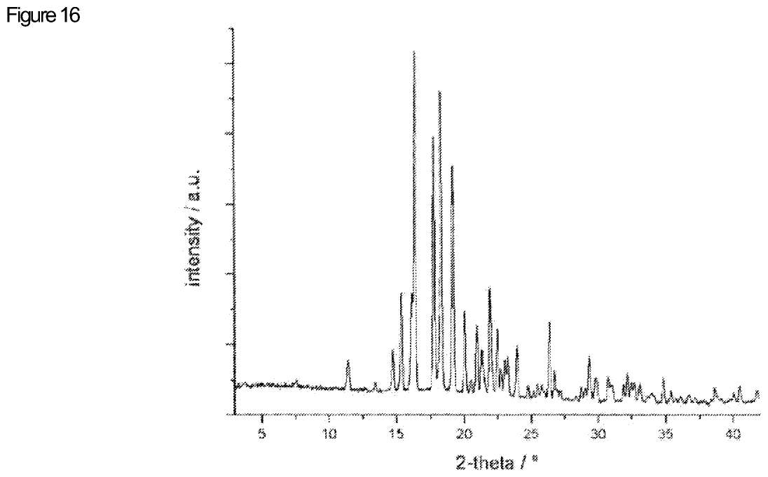

In certain embodiments, form B can be characterized by the X-ray diffraction pattern as substantially shown on FIG. 16.

In certain embodiments, form B can be characterized by an infrared spectrum having sharp bands at: 1644, 1635, 1621, 1599, 1567, 1514, 1488, 1398, 1343, 1328, 1291, 1266, 1183, 1155, 1090, 1022, 1003, 973, 958, 938, 920, 897, 822, 783, 753, 740, 683 and 638 cm⁻¹ (+3 cm⁻¹).

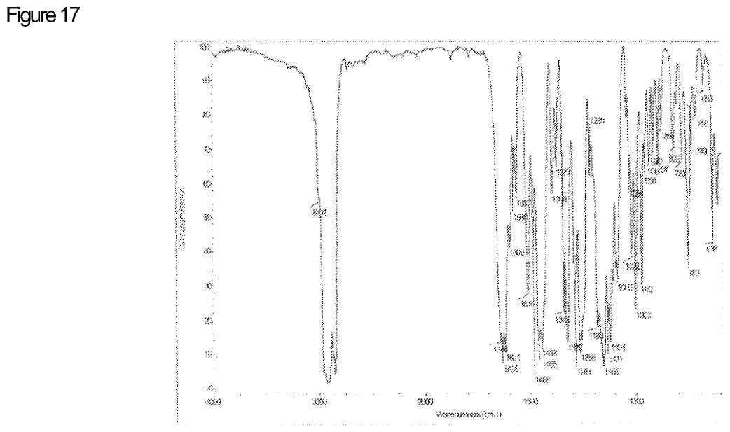

In certain embodiments, form B can be characterized by an infrared spectrum as substantially shown in FIG. 17.

In certain embodiments, form B can be characterized by a melting point with onset temperature (DSC) in the range of about 151° C. to 154° C.

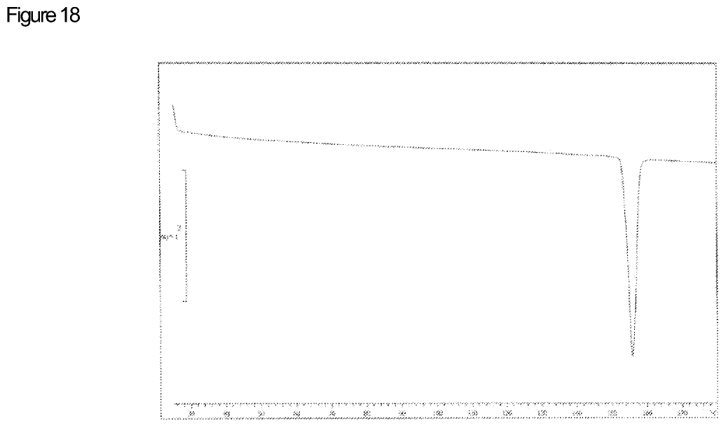

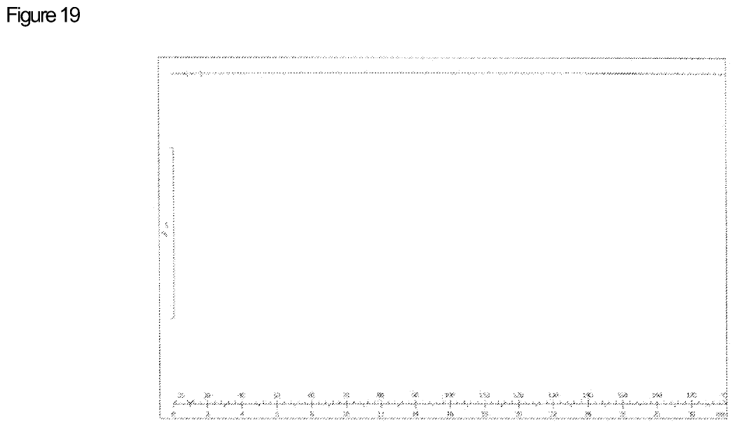

In certain embodiments, certain characteristics of form B are shown on FIGS. 16 to 19.

A single crystal structure analysis of form B was conducted. Table 2 lists the some crystal structure data. The experimental XRPD pattern collected with the form B corresponds to the theoretical pattern calculated from crystal structure data. In the single crystal structure of form B the piperazine ring shows chair conformation with the pyridine substituent standing in axial position.

TABLE 2
Crystal structure data for the form B crystal

| Name | Form B |
|---|---|
| Empirical Formula | C₂₁H₂₀F₇N₃O₄S |
| Formula Weight | 543.46 g · mol⁻¹ |
| Temperature | 88K |
| Space Group | P2(1) |
| Unit Cell Dimensions | A = 16.420(3) A |
| | alpha = 90 deg. |
| | B = 6.1000(12) A |
| | beta = 106.49(3) deg. |
| | C = 23.750(5) A |
| | gamma = 90 deg. |
| Cell Volume | 2281.0(8) A³ |
| Molecules in Unit Cell | 4 |
| Calculated Density | 1.583 g/cm³ |

In some embodiments of the methods or uses described herein, the compound [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone comprises at least 70% of a crystalline polymorph of form B; in certain embodiments, it comprises at least 90% of a crystalline polymorph of form B; in certain embodiments, it comprises at least 96% of a crystalline polymorph of form B; in certain embodiments, it comprises at least 99% of a crystalline polymorph of form B.

In some embodiments, the methods or uses described herein relate to form C. In certain embodiments, form C can be prepared by a method comprising the step of:
crystallization of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone; or
crystallization of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone by seeding with form C; or
tempering of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone at about 150° C. and subsequent cooling.

In certain embodiments, form C can be obtained by crystallization from a toluene or toluene/n-heptane solution at 100° C. In certain embodiments, form C can be prepared by crystallization of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone in several solvents and seeding with form C. In certain embodiments, form C can be obtained by tempering of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone at 150° C. for 2 hours and subsequent rapid cooling. Certain aspects of the methods of preparation and in particular, the preparation of seeding crystals are described in the examples of this application.

In certain embodiments, form C is a solvent-free form as no significant weight loss is observed in the TGA curve prior to decomposition.

In certain embodiments, form C can be characterized by at least three peaks selected from the following X-ray diffraction peaks obtained with a CuKa radiation expressed in degrees 2Theta at approximately: 14.9, 15.7, 16.7, 17.7, 17.8, 18.7, 19.7, 21.8, 22.0, 25.2.

In certain embodiments, form C can be characterized by at least five peaks selected from the following X-ray diffraction peaks obtained with a CuKa radiation expressed in degrees 2Theta at approximately: 14.9, 15.7, 16.7, 17.7, 17.8, 18.7, 19.7, 21.8, 22.0, 25.2.

In certain embodiments, form C can be characterized by at least seven peaks selected from the following X-ray diffraction peaks obtained with a CuKa radiation expressed in degrees 2Theta at approximately: 14.9, 15.7, 16.7, 17.7, 17.8, 18.7, 19.7, 21.8, 22.0, 25.2. In certain embodiments, form C can also be characterized by the following X-ray diffraction peaks obtained with a CuKa radiation expressed in degrees 2Theta at approximately: 14.9, 15.7, 16.7, 17.7, 17.8, 18.7, 19.7, 21.8, 22.0 and 25.2.

The term “approximately” means in this context that there is an uncertainty in the measurements of the degrees 2Theta of +0.2 (expressed in degrees 2Theta).

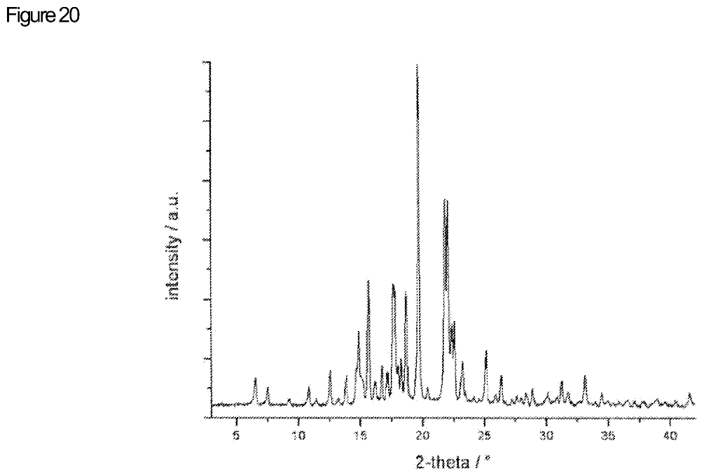

In certain embodiments, form C can be characterized by the X-ray diffraction pattern as substantially shown on FIG. 20.

In certain embodiments, form C can be characterized by an infrared spectrum having sharp bands at: 1641, 1622, 1601, 1581, 1566, 1514, 1398, 1378, 1341, 1322, 1309, 1294, 1281, 1159, 1087, 1023, 1009, 966, 934, 917, 901, 822, 784, 757, 681 and 640 cm⁻¹ (+3 cm-1).

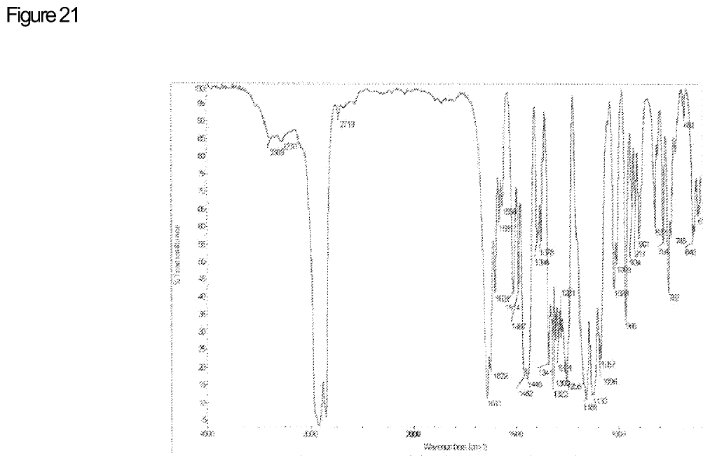

In certain embodiments, form C can be characterized by infrared spectrum as substantially shown on FIG. 21.

In certain embodiments, form C can also be characterized by a melting point with onset temperature (DSC) in the range of about 152° C. to 156° C.

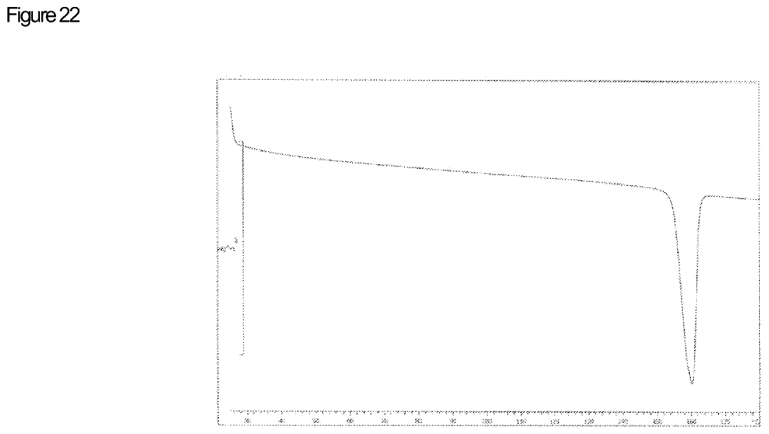

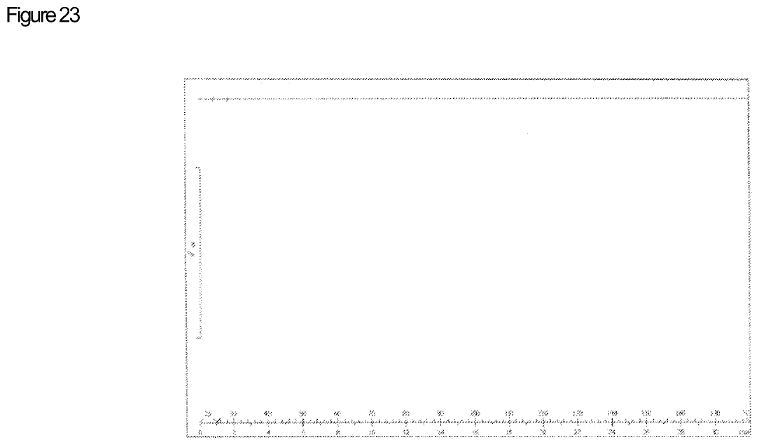

Certain characteristics of form C are shown on FIGS. 20 to 23.

In certain embodiments of the methods or uses of this application, the compound, [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone comprises at least 70% of a crystalline polymorph of form C; in certain embodiments, it comprises at least 90% of a crystalline polymorph of form C; in certain embodiments, it comprises at least 96% of a crystalline polymorph of form C; in certain embodiments, it comprises at least 99% of a crystalline polymorph of form C.

In some embodiments, the methods or uses described herein relate to an amorphous form of Bitopertin. In certain embodiments, an amorphous form of Bitopertin can be prepared by a method comprising the step of:
fast evaporation from a solution of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone at about 40° C. under vacuum; or
lyophilization of a solution of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone.

In some embodiments, the amorphous form can be obtained from an ethanol solution upon fast evaporation at about 40° C. under vacuum. In some embodiments, the amorphous form can be obtained by lyophilization of a solution of 1.0 g of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone in 50 mL acetonitrile (condensator at −46° C. and vacuum at 0-1 mbar). Certain aspects of the methods of preparation of the amorphous form are further described in the examples of this application.

In some embodiments, the amorphous form can be characterized by the lack of sharp X-ray diffraction peaks in its XRPD pattern.

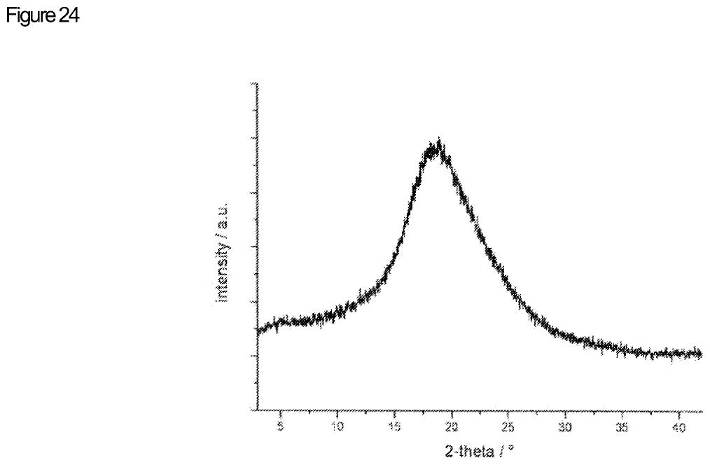

In some embodiments, the amorphous form can be characterized by the X-ray diffraction pattern as substantially shown on FIG. 24.

In some embodiments, the amorphous form can be characterized by an infrared spectrum having sharp bands at 1642, 1622, 1599, 1579, 1509, 1487, 1399, 1329, 1293, 1253, 1159, 1124, 1090, 1016, 960, 920, 903, 889, 827, 782, 763, 739 and 636 cm⁻¹ (+3 cm⁻¹).

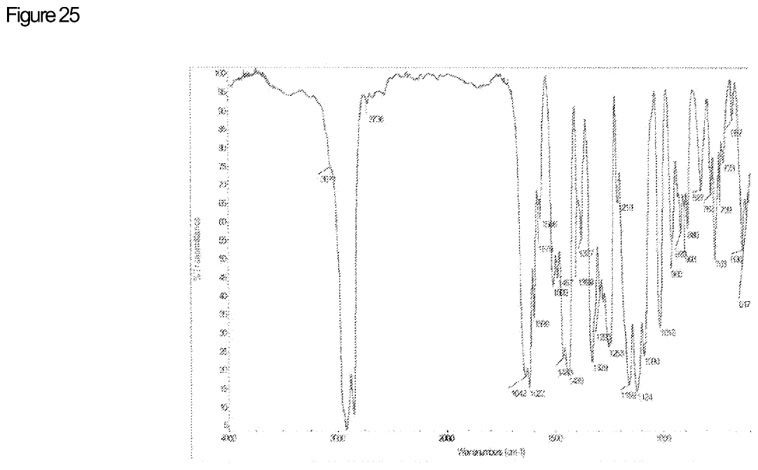

In some embodiments, the amorphous form can be characterized by infrared spectrum as substantially shown on FIG. 25.

In some embodiments, the amorphous form can be characterized by a glass transition temperature (DSC, heating rate 10 K/min, closed pan) of about 48° C. to about 65° C. (in some embodiments, the glass transition temperature is largely dependent on the solvent/water content).

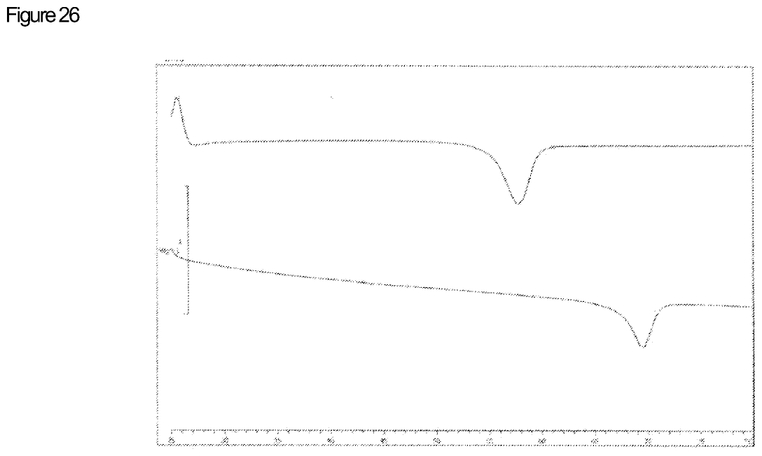

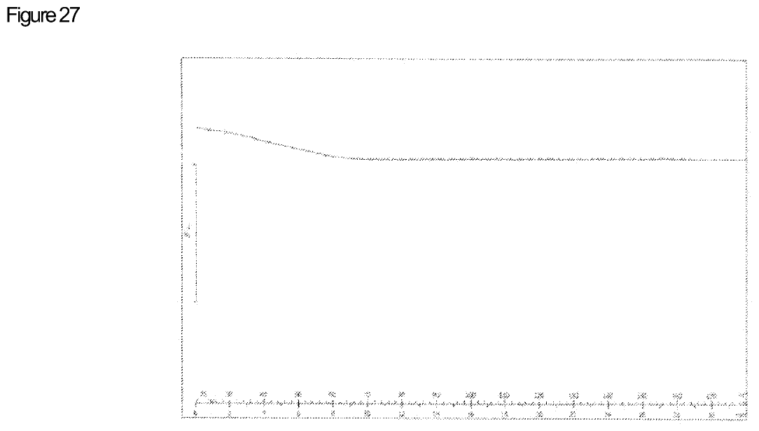

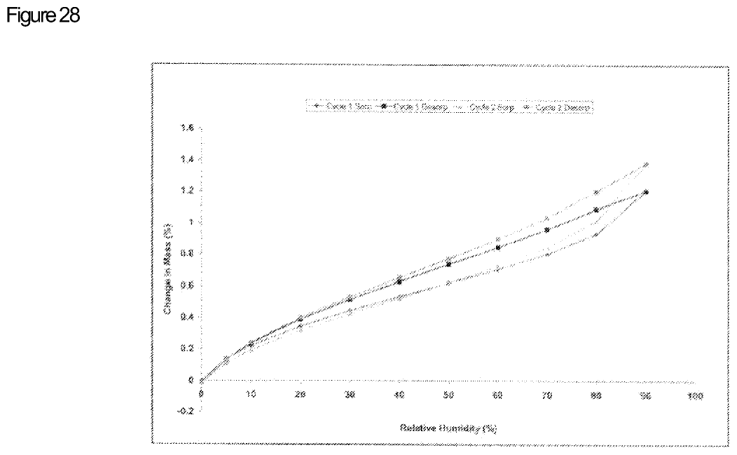

Certain characteristics of the amorphous form of Bitopertin are shown on FIGS. 24 to 28.

In some embodiments of the application, the compound [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone comprises at least 70% of an amorphous form; in certain embodiments, it comprises at least 90% of an amorphous form; in certain embodiments, it comprises at least 96% of an amorphous form; in certain embodiments, it comprises at least 99% of an amorphous form.

In certain embodiments of the application, the methylparaben cocrystal form can be prepared by a method comprising the steps of re-crystallization of form A, B, C or amorphous form and methylparaben, with or without seeding in solvent systems.

In some embodiments, the methods or uses of this application relates to a methylparaben cocrystal of Bitopertin. In certain embodiments, the methylparaben cocrystal form can be produced by digestion in solvents, such as ethanol and water. In certain embodiments, the methylparaben cocrystal form can be prepared by re-crystallization of form A, B, C or amorphous form and methylparaben, with or without seeding in a solvent system comprising but not limited to ethanol. In certain embodiments, the ratio of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone and methylparaben in the cocrystal form ranges from 1:1 to 1:10. In some embodiments, the ratio of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone and methylparaben in the cocrystal is 1:1.

Certain aspects of the methods of preparation and in particular the preparation of seeding crystals are further elucidated in the examples of this application.

In certain embodiments, the methylparaben cocrystal form can be characterized by at least three peaks selected from the following X-ray diffraction peaks obtained with a CuKa radiation expressed in degrees 2Theta at approximately: 8.0, 8.9, 10.5, 12.6, 15.2, 16.1, 17.7, 18.5, 19.8, 20.2, 21.7, 22.9, 24.2, 25.9.

In certain embodiments, the methylparaben cocrystal form can be characterized by at least five peaks selected from the following X-ray diffraction peaks obtained with a CuKa radiation expressed in degrees 2Theta at approximately: 8.0, 8.9, 10.5, 12.6, 15.2, 16.1, 17.7, 18.5, 19.8, 20.2, 21.7, 22.9, 24.2, 25.9.

In certain embodiments, the methylparaben cocrystal form can be characterized by at least seven peaks selected from the following X-ray diffraction peaks obtained with a CuKa radiation expressed in degrees 2Theta at approximately: 8.0, 8.9, 10.5, 12.6, 15.2, 16.1, 17.7, 18.5, 19.8, 20.2, 21.7, 22.9, 24.2, 25.9.

In certain embodiments, the methylparaben cocrystal form can be characterized by the following X-ray diffraction pattern obtained with a CuKa radiation expressed in degrees 2Theta at approximately: 8.0, 8.9, 10.5, 12.6, 15.2, 16.1, 17.7, 18.5, 19.8, 20.2, 21.7, 22.9, 24.2 and 25.9.

The term “approximately” means in this context that there is an uncertainty in the measurements of the degrees 2Theta of ±0.2 (expressed in degrees 2Theta).

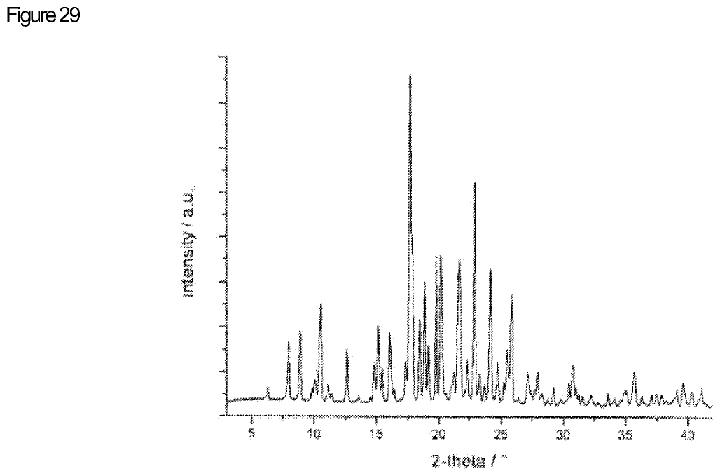

In certain embodiments, the methylparaben cocrystal form can also be characterized by the X-ray diffraction pattern as substantially shown on FIG. 29.

In certain embodiments, the methylparaben cocrystal form can also be characterized by an infrared spectrum having sharp bands at 3154, 3081, 1709, 1614, 1586, 1378, 1337, 1313, 1247, 1189, 1172, 1124, 1085, 1019, 959, 928, 916, 908, 894, 857, 783, 772, 729 and 702 cm⁻¹ (+3 cm⁻¹).

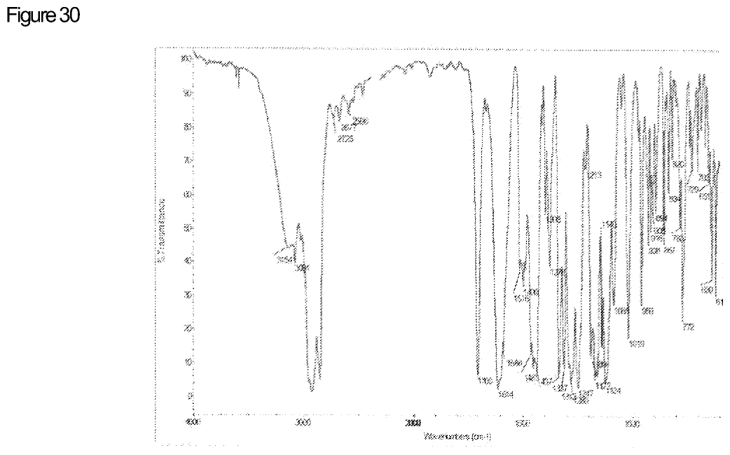

In certain embodiments, the methylparaben cocrystal form can be characterized by the infrared spectrum as substantially shown on FIG. 30.

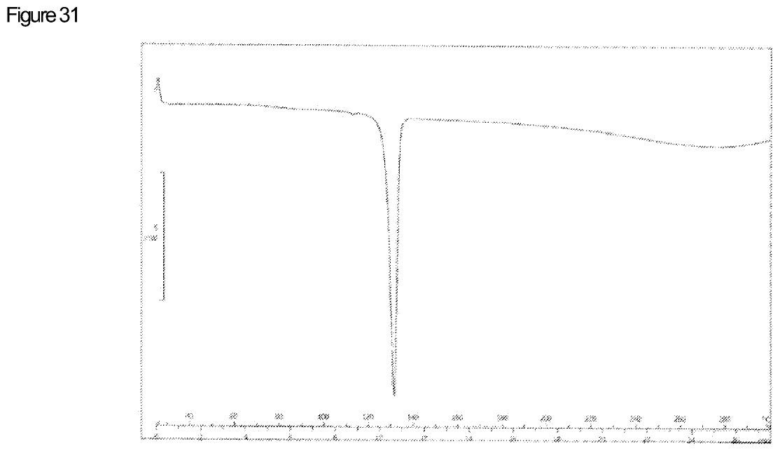

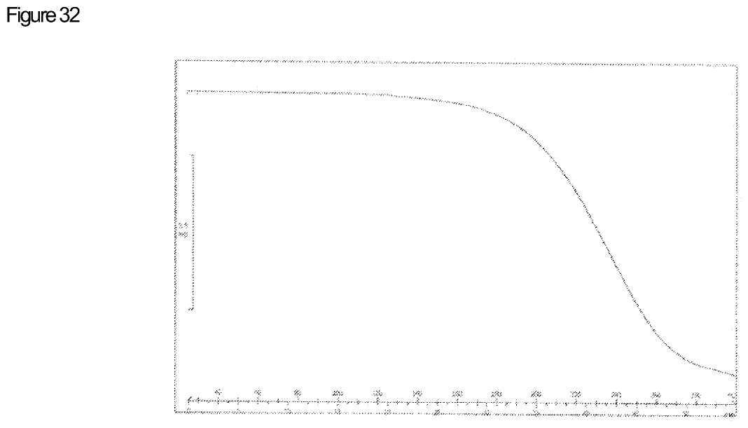

Certain characteristics of the methylparaben cocrystal form of Bitopertin are shown in FIGS. 29 to 32.

A single crystal structure analysis of the methylparaben cocrystal was conducted. Table 3 lists some crystal structure data. The experimental XRPD pattern collected with the methylparaben cocrystal corresponds to the theoretical pattern calculated from crystal structure data.

TABLE 3
Crystal structure data for the methylparaben cocrystal form.

| Name | methylparaben cocrystal form |
|---|---|
| Empirical Formula | C₂₉H₂₈F₇N₃O₇S |
| Formula Weight | 695.60 g · mol⁻¹ |
| Temperature | 89K |
| Space Group | P1 |
| Unit Cell Dimensions | a = 10.140(2) A |
| | alpha = 83.65(3) deg. |
| | B = 11.690(2) A |
| | beta = 79.88(3) deg. |
| | C = 13.870(3) A |
| | gamma = 72.75(3) deg. |
| Cell Volume | 1542.8(5) A³ |
| Molecules in Unit Cell | 2 |
| Calculated Density | 1.497 g/cm³ |

In certain embodiments of the application, the compound comprises at least 70% of a methylparaben cocrystal of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone; in certain embodiments, it comprises at least 90% of a methylparaben cocrystal; in certain embodiments, it comprises at least 96% of a methylparaben cocrystal; in certain embodiments, it comprises at least 99% of a methylparaben cocrystal.

In certain embodiments of the methods and uses disclosed herein, the subject is a subject in need thereof.

In some embodiments of the uses and methods as disclosed herein, the glycine transporter inhibitor, such as a GlyT1 inhibitor (e.g., a crystalline or amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone as described herein), is administered in a therapeutically effective amount.

In certain embodiments, a crystalline or amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone as described herein can be used to inhibit the GlyT1 transporter. Thus, in some embodiments, the crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein can be referred to as GlyT1 transporter inhibiting compounds or GlyT1 inhibitors.

The crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein can be administered in any conventional manner by any route where they are active. Administration can be systemic, topical, or oral. For example, administration can be, but is not limited to, parenteral, subcutaneous, intravenous, intramuscular, intraperitoneal, transdermal, oral, buccal, sublingual, or ocular routes, or intravaginal, by inhalation, by depot injections, or by implants. The mode of administration can depend on the conditions or disease to be targeted or treated. The selection of the specific route of administration can be selected or adjusted by the clinician according to methods known to the clinician to obtain the desired clinical response.

In some embodiments, it may be desirable to administer a crystalline or amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone locally to an area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, wherein the implant is of a porous, non-porous, or gelatinous material, including membranes, such as silastic membranes, or fibers.

The crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein can be administered either alone or in combination (concurrently or serially) with other pharmaceuticals. For example, a crystalline or amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein can be administered in combination with other drugs for the treatment of EPP, XLPP, or CEP and the like. Examples of other pharmaceuticals or medicaments are known to one of skill in the art and include, but are not limited to those described herein.

The means and methods for administration are known in the art and an artisan can refer to various pharmacologic references for guidance (see, for example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980)).

The amount of the crystalline or amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician). The standard dosing for protamine can be used and adjusted (i.e., increased or decreased) depending upon the factors described herein. The selection of the specific dose regimen can be selected or adjusted or titrated by the clinician according to methods known to the clinician to obtain the desired clinical response.

The amount of a crystalline or amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein that will be effective in the treatment and/or prevention of a particular disease, condition, or disorder will depend on the nature and extent of the disease, condition, or disorder, and can be determined by standard clinical techniques. In addition, in vitro or in vivo assays may optionally be employed to help identify optimal dosage ranges. The precise dose to be employed in the compositions will also depend on the route of administration, and the seriousness of the disorder, and should be decided according to the judgment of the practitioner and each patient's circumstances. However, a suitable dosage range for oral administration is, generally, from about 0.001 milligram to about 200 milligrams per kilogram body weight, from about 0.01 milligram to about 100 milligrams per kilogram body weight, from about 0.01 milligram to about 70 milligrams per kilogram body weight, from about 0.1 milligram to about 50 milligrams per kilogram body weight, from 0.5 milligram to about 20 milligrams per kilogram body weight, or from about 1 milligram to about 10 milligrams per kilogram body weight. In some embodiments, the oral dose is about 5 milligrams per kilogram body weight. In some embodiments of oral administration, the dosage for adults can vary from about 0.01 mg to about 1000 mg, from about 1 mg to about 240 mg, or from about 3 mg to about 120 mg per day.

In some embodiments, suitable dosage ranges for intravenous (i.v.) administration are from about 0.01 mg to about 500 mg per kg body weight, from about 0.1 mg to about 100 mg per kg body weight, from about 1 mg to about 50 mg per kg body weight, or from about mg to about 35 mg per kg body weight. Suitable dosage ranges for other modes of administration can be calculated based on the forgoing dosages as known by those skilled in 10 the art. For example, recommended dosages for intranasal, transmucosal, intradermal, intramuscular, intraperitoneal, subcutaneous, epidural, sublingual, intracerebral, intravaginal, transdermal administration or administration by inhalation are in the range of from about 0.001 mg to about 200 mg per kg of body weight, from about 0.01 mg to about 100 mg per kg of body weight, from about 0.1 mg to about 50 mg per kg of body weight, or from about 1 mg to about 20 mg per kg of body weight. Effective doses may be extrapolated from dose-response curves derived from in vitro or animal model test systems. Such animal models and systems are well known in the art.

The crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein can be formulated for parenteral administration by injection, such as by bolus injection or continuous infusion. In some embodiments, the crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone can be administered by continuous infusion subcutaneously over a period of about 15 minutes to about 24 hours. Formulations for injection can be presented in unit dosage form, such as in ampoules or in multi-dose containers, with an optionally added preservative. The compositions can take such forms as suspensions, or emulsions in oily or aqueous vehicles, and can contain formulatory agents such as suspending, stabilizing and/or dispersing agents. In some embodiments, the injectable is in the form of short-acting, depot, or implant and pellet forms injected subcutaneously or intramuscularly. In some embodiments, the parenteral dosage form is the form of a suspension, emulsion, or dry powder.

For oral administration, the crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein can be formulated by combining the crystalline or amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone with pharmaceutically acceptable carriers well known in the art. Such carriers enable the compounds to be formulated as tablets, pills, dragees, capsules, emulsions, liquids, gels, syrups, caches, pellets, powders, granules, slurries, lozenges, aqueous or oily suspensions, and the like, for oral ingestion by a patient to be treated. Pharmaceutical preparations for oral use can be obtained by, for example, adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds. Oral compositions can include standard vehicles such as mannitol, lactose, starch, magnesium stearate, sodium saccharine, cellulose, magnesium carbonate, etc. Such vehicles are suitably of pharmaceutical grade.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added.

For buccal administration, the compositions can take the form of, such as, tablets or lozenges formulated in a conventional manner.

For administration by inhalation, the crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein can be delivered in the form of an aerosol spray presentation from pressurized packs or a nebulizer, with the use of a suitable propellant, such as dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, such as gelatin for use in an inhaler or insufflator can be formulated containing a powder mix of the compound and a suitable powder base such as lactose or starch.

The crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein can also be formulated in rectal compositions such as suppositories or retention enemas, such as containing conventional suppository bases such as cocoa butter or other glycerides. The crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein can also be formulated in vaginal compositions such as vaginal creams, suppositories, pessaries, vaginal rings, and intrauterine devices.

In transdermal administration, the crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone can be applied to a plaster, or can be applied by transdermal, therapeutic systems that are consequently supplied to the organism. In some embodiments, the crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone are present in creams, powders, fluid emulsions, fluid suspensions, semi-solids, ointments, pastes, gels, jellies, and foams, or in patches containing any of the same.

The crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein can also be formulated as a depot preparation. Such long acting formulations can be administered by implantation (for example subcutaneously or intramuscularly) or by intramuscular injection. Depot injections can be administered at about 1 to about 6 months or longer intervals. Thus, for example, the crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone can be formulated with suitable polymeric or hydrophobic materials (for example as an emulsion in an acceptable oil) or ion exchange resins, or as sparingly soluble derivatives, for example, as a sparingly soluble salt.

In some embodiments, the crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone can be delivered in a controlled release system. In one embodiment, a pump may be used (see Langer, supra; Sefton, CRC Crit. Ref. Biomed. Eng., 1987, 14, 201; Buchwald et al., Surgery, 1980, 88, 507 Saudek et al., N. Engl. J. Med., 1989, 321, 574). In some embodiments, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger et al., J. Macromol. Sci. Rev. Macromol. Chem., 1983, 23, 61; see, also Levy et al., Science, 1985, 228, 190; During et al., Ann. Neurol., 1989, 25, 351; Howard et al., J. Neurosurg., 1989, 71, 105). In yet another embodiment, a controlled-release system can be placed in proximity of the target of the crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein, such as the liver, thus requiring only a fraction of the systemic dose (see, e.g., Goodson, in Medical Applications of Controlled Release, supra, vol. 2, p p. 115-138 (1984)). Other controlled-release systems discussed in the review by Langer, Science, 1990, 249, 1527-1533) may be used.

In certain embodiments, the crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The pharmaceutical compositions can also comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols. In some embodiments, the compounds described herein can be used with agents including, but not limited to, topical analgesics (e.g., lidocaine), barrier devices (e.g., GelClair), or rinses (e.g., Caphosol).

In some embodiments, the crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein can be delivered in a vesicle, in particular a liposome (see, Langer, Science, 1990, 249, 1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

Suitable compositions include, but are not limited to, oral non-absorbed compositions. Suitable compositions also include, but are not limited to saline, water, cyclodextrin solutions, and buffered solutions of pH 3-9.

The crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein can be formulated with numerous excipients including, but not limited to, purified water, propylene glycol, PEG 400, glycerin, DMA, ethanol, benzyl alcohol, citric acid/sodium citrate (pH3), citric acid/sodium citrate (pH5), tris(hydroxymethyl) amino methane HCl (pH7.0), 0.9% saline, and 1.2% saline, and any combination thereof. In some embodiments, excipient is chosen from propylene glycol, purified water, and glycerin.

In some embodiments, the formulation can be lyophilized to a solid and reconstituted with, for example, water prior to use.

When administered to a mammal (e.g., to an animal for veterinary use or to a human for clinical use) the crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone can be administered in isolated form.

When administered to a human, the crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone can be sterile. Water is a suitable carrier when the crystalline or amorphous form(s) of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The compositions described herein can take the form of a suspension, emulsion, tablet, pill, pellet, capsule, capsule containing a liquid, powder, sustained-release formulation, suppository, aerosol, spray, or any other form suitable for use. Examples of suitable pharmaceutical carriers are described in Remington's Pharmaceutical Sciences, A. R. Gennaro (Editor) Mack Publishing Co.

In some embodiments, the crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone are formulated in accordance with routine procedures as a pharmaceutical composition adapted for administration to humans. Typically, compounds are solutions in sterile isotonic aqueous buffer. Where necessary, the compositions can also include a solubilizing agent. Compositions for intravenous administration may optionally include a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the crystalline or amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone as described herein is to be administered by infusion, it can be dispensed, for example, with an infusion bottle containing sterile pharmaceutical grade water or saline. Where a crystalline or amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone as described herein is administered by injection, an ampoule of sterile water for injection or saline can be provided so that the ingredients may be mixed prior to administration.

The pharmaceutical compositions can be in unit dosage form. In such form, the composition can be divided into unit doses containing appropriate quantities of the active component. The unit dosage form can be a packaged preparation, the package containing discrete quantities of the preparations, for example, packeted tablets, capsules, and powders in vials or ampules. The unit dosage form can also be a capsule, cachet, or tablet itself, or it can be the appropriate number of any of these packaged forms. Table 4 provides an example of a typical capsule formulation, which can be prepared according to the application.

TABLE 4
Formulation Capsule Formulation (Wet granulation)

| | | mg/capsule | | | | |
|---|---|---|---|---|---|---|
| Item | Ingredients | 1.0 mg | 3.0 mg | 10.0 mg | 25.0 mg | 40.0 mg |
| 1 | Form A of active ingredient | 1.00 | 3.00 | 10.00 | 25.00 | 40.00 |
| 2 | Lactose Monohydrate | 114.00 | 112.00 | 105.00 | 90.00 | 75.00 |
| 3 | Maize Starch | 60.00 | 60.00 | 60.00 | 60.00 | 60.00 |
| 4 | Sodium Starch Glycolate | 10.00 | 10.00 | 10.00 | 10.00 | 10.00 |
| 5 | Povidone 30 | 10.00 | 10.00 | 10.00 | 10.00 | 10.00 |
| 6 | Talc | 4.00 | 4.00 | 4.00 | 4.00 | 4.00 |
| 7 | Magnesium Stearate | 1.00 | 1.00 | 1.00 | 1.00 | 1.00 |
| | Total | 200.00 | 200.00 | 200.00 | 200.00 | 200.00 |

An exemplary manufacturing process for making a capsule with the ingredients of Table 4 comprises the following steps:
1. Mix items 1, 2, 3, 4 and 5 in a suitable mixer;
2. Granulate the mixed powder from step 1 with granulation liquid;
3. Screen the mixture received from step 2, dry and sieve the granules;
4. Add items 6 and 7 to the dried and sieved granules from step 3 and mix; and
5. Fill mixture from step 4 into a suitable capsule.

In some embodiments, a composition is in the form of a liquid wherein the active agent is present in suspension, as an emulsion. In some embodiments, the liquid composition is in the form of a gel. In other embodiments, the liquid composition is aqueous. In other embodiments, the composition is in the form of an ointment.

In some embodiments, the composition is in the form of a solid article. For example, in some embodiments, the ophthalmic composition is a solid article that can be inserted in a suitable location in the eye, such as between the eye and eyelid or in the conjunctival sac, where it releases the active agent as described, for example, U.S. Pat. Nos. 3,863,633; 3,867,519; 3,868,445; 3,960,150; 3,963,025; 4,186,184; 4,303,637; 5,443,505; and 5,869,079. Release from such an article is usually to the cornea, either via the lacrimal fluid that bathes the surface of the cornea, or directly to the cornea itself, with which the solid article is generally in intimate contact. Solid articles suitable for implantation in the eye in such fashion are generally composed primarily of polymers and can be bioerodible or non-bioerodible. Bioerodible polymers that can be used in the preparation of ocular implants carrying one or more of compounds include, but are not limited to, aliphatic polyesters such as polymers and copolymers of poly(glycolide), poly(lactide), poly(epsilon-caprolactone), poly-(hydroxybutyrate) and poly(hydroxyvalerate), polyamino acids, polyorthoesters, polyanhydrides, aliphatic polycarbonates and polyether lactones. Suitable non-bioerodible polymers include silicone elastomers.

The compositions described herein can contain preservatives. Suitable preservatives include, but are not limited to, mercury-containing substances such as phenylmercuric salts (e.g., phenylmercuric acetate, borate and nitrate) and thimerosal; stabilized chlorine dioxide; quaternary ammonium compounds such as benzalkonium chloride, cetyltrimethylammonium bromide and cetylpyridinium chloride; imidazolidinyl urea; parabens such as methylparaben, ethylparaben, propylparaben and butylparaben, and salts thereof; phenoxyethanol; chlorophenoxyethanol; phenoxypropanol; chlorobutanol; chlorocresol; phenylethyl alcohol; disodium EDTA; and sorbic acid and salts thereof.

Optionally one or more stabilizers can be included in the compositions to enhance chemical stability where required. Suitable stabilizers include, but are not limited to, chelating agents or complexing agents, such as, for example, the calcium complexing agent ethylene diamine tetraacetic acid (EDTA). For example, an appropriate amount of EDTA or a salt thereof, e.g., the disodium salt, can be included in the composition to complex excess calcium ions and prevent gel formation during storage. EDTA or a salt thereof can suitably be included in an amount of about 0.01% to about 0.5%. In those embodiments containing a preservative other than EDTA, the EDTA or a salt thereof, more particularly disodium EDTA, can be present in an amount of about 0.025% to about 0.1% by weight.

One or more antioxidants can also be included in the compositions. Suitable antioxidants include, but are not limited to, ascorbic acid, sodium metabisulfite, sodium bisulfite, acetylcysteine, polyquaternium-1, benzalkonium chloride, thimerosal, chlorobutanol, methyl paraben, propyl paraben, phenylethyl alcohol, edetate disodium, sorbic acid, or other agents know to those of skill in the art. Such preservatives are typically employed at a level of from about 0.001% to about 1.0% by weight.

In some embodiments, the crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein are solubilized at least in part by an acceptable solubilizing agent. Certain acceptable nonionic surfactants, for example polysorbate 80, can be useful as solubilizing agents, as can ophthalmically acceptable glycols, polyglycols, e.g., polyethylene glycol 400 (PEG-400), and glycol ethers.

Suitable solubilizing agents are cyclodextrins. Suitable cyclodextrins can be chosen from α-cyclodextrin, β-cyclodextrin, γ-cyclodextrin, alkylcyclodextrins (e.g., methyl-β-cyclodextrin, dimethyl-β-cyclodextrin, diethyl-β-cyclodextrin), hydroxyalkylcyclodextrins (e.g., hydroxyethyl-β-cyclodextrin, hydroxypropyl-β-cyclodextrin), carboxy-alkylcyclodextrins (e.g., carboxymethyl-β-cyclodextrin), sulfoalkylether cyclodextrins (e.g., sulfobutylether-β-cyclodextrin), and the like. Ophthalmic applications of cyclodextrins have been reviewed in Rajewski et al., Journal of Pharmaceutical Sciences, 1996, 85, 1155-1159.

In some embodiments, the composition optionally contains a suspending agent. For example, in those embodiments in which the composition is an aqueous suspension or solution/suspension, the composition can contain one or more polymers as suspending agents. Useful polymers include, but are not limited to, water-soluble polymers such as cellulosic polymers, for example, hydroxypropyl methylcellulose, and water-insoluble polymers such as cross-linked carboxyl-containing polymers.

One or more acceptable pH adjusting agents and/or buffering agents can be included in the compositions, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Optionally one or more acceptable surfactants, such as, but not limited to, nonionic surfactants, or co-solvents can be included in the compositions to enhance solubility of the components of the compositions or to impart physical stability, or for other purposes. Suitable nonionic surfactants include, but are not limited to, polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40; polysorbate 20, 60 and 80; polyoxyethylene/polyoxypropylene surfactants (e.g., Pluronic® F-68, F84 and P-103); cyclodextrin; or other agents known to those of skill in the art. Typically, such co-solvents or surfactants are employed in the compositions at a level of from about 0.01% to about 2% by weight.

In some embodiments, pharmaceutical packs or kits comprising one or more containers filled with one or more crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein are provided. Optionally associated with such container(s) can be a notice in the form prescribed by a governmental agency regulating the manufacture, use or sale of pharmaceuticals or biological products, which notice reflects approval by the agency of manufacture, use or sale for human administration for treating a condition, disease, or disorder described herein. In some embodiments, the kit contains more than one crystalline or amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein. In some embodiments, the kit comprises a crystalline or amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein in a single injectable dosage form, such as a single dose within an injectable device such as a syringe with a needle.

In some embodiments, the methods comprise administering to the subject one or more crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein or a pharmaceutical composition of the same. In some embodiments, the subject is a subject in need of such treatment. As described herein, in some embodiments, the subject is a mammal, such as, but not limited to, a human.

In some embodiments, also provided are one or more crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein, or a pharmaceutical composition comprising one or more crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein, for use in the manufacture of a medicament for the treatment of methods of treating and/or preventing EPP, XLPP, or CEP, or related syndrome thereof, including, but not limited to the conditions described herein, in a subject, such as those described herein. In some embodiments, the subject is a subject in need thereof.

The present embodiments also provides the use of one or more crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein, or a pharmaceutical composition comprising one or more crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein, in the inhibition of a GlyT1 transporter, such as the presence on the surface of the cell. In some embodiments, the crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone, or a pharmaceutical composition of the same inhibit the internalization, trafficking, and/or degradation of the GlyT1 transporter.

As used herein, “inhibition” can refer to either inhibition of a specific activity. The activity of a GlyT1 transporter can be measured by any method known in the art including but not limited to the methods described herein.

The crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein are inhibitors of the GlyT1 transporter. The ability of the crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone to inhibit GlyT1 transporter activity may be measured using any assay known in the art.

Generally, assays for testing compounds that inhibit GlyT1 transporter activity include the determination of any parameter that is indirectly or directly under the influence of a GlyT1 transporter, e.g., a functional, physical, or chemical effect. Samples or assays comprising GlyT1 transporters that are treated with a potential inhibitor, are compared to control samples without the inhibitor to examine the extent of inhibition. Control samples (untreated with inhibitors) are assigned a relative GlyT1 transporter activity value of 100%. Inhibition of a GlyT1 transporter is achieved when the GlyT1 transporter activity value relative to the control is about 80%, 50%, or 25%.

Ligand binding to a GlyT1 transporter can be tested in a number of formats. Binding can be performed in solution, in a bilayer membrane, attached to a solid phase, in a lipid monolayer, or in vesicles. For example, in an assay, the binding of the natural ligand to its transporter is measured in the presence of a candidate modulator, such as the crystalline or amorphous form(s) of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein. Alternatively, the binding of the candidate modulator may be measured in the presence of the natural ligand. Often, competitive assays that measure the ability of a compound to compete with binding of the natural ligand to the transporter are used. Binding can be tested by measuring, e.g., changes in spectroscopic characteristics (e.g., fluorescence, absorbance, refractive index), hydrodynamic (e.g., shape) changes, or changes in chromatographic or solubility properties.

After the transporter is expressed in cells, the cells can be grown in appropriate media in the appropriate cell plate. The cells can be plated, for example at 5000-10000 cells per well in a 384 well plate. In some embodiments, the cells are plated at about 1000, 2000, 3000, 4000, 5000, 6000, 7000, 8000, 9000, or 10000 cells/per well. The plates can have any number of wells and the number of cells can be modified accordingly.

Any medicament having utility in an application described herein can be used in co-therapy, co-administration or co-formulation with a composition as described herein. Therefore, the crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein can be administered either before, concurrently with, or after such therapeutics are administered to a subject.

The additional medicament can be administered in co-therapy (including co-formulation) with the one or more of the crystalline or amorphous forms of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein.

In some embodiments, the response of the disease or disorder to the treatment is monitored and the treatment regimen is adjusted if necessary in light of such monitoring.

Frequency of administration is typically such that the dosing interval, for example, the period of time between one dose and the next, during waking hours is from about 1 to about 24, about 2 to about 12 hours, from about 3 to about 8 hours, or from about 4 to about 6 hours. In some embodiments, the dose is administered 1, 2, 3, or 4 times a day. It will be understood by those of skill in the art that an appropriate dosing interval is dependent to some degree on the length of time for which the selected composition is capable of maintaining a concentration of the compound, e.g., a crystalline or amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone described herein, in the subject and/or in the target tissue (e.g., above the EC50 (the minimum concentration of the compound which inhibits the transporter's activity by 90%). Ideally, the concentration remains above the EC50 for at least 100% of the dosing interval. Where this is not achievable it is desired that the concentration should remain above the EC50 for at least about 60% of the dosing interval or should remain above the EC₅₀ for at least about 40% of the dosing interval.

### METHODS OF USE

The present application provides methods of preventing or treating disorders associated with accumulation of PPIX in a subject, the method comprising administering to the subject one or more crystalline or amorphous form(s) of Bitopertin as described herein. In part, the present disclosure relates to methods of treating erythropoietic protoporphyria (EPP), X-linked protoporphyria (XLPP), or congenital erythropoietic porphyria (CEP) in a subject, the method comprising administering to the subject a pharmaceutical composition comprising one or more crystalline or amorphous forms of Bitopertin as described herein. In certain embodiments, the present disclosure provides methods of preventing, treating, or reducing the progression rate and/or severity of one or more complications of EPP, XLPP, or CEP in a subject, the method comprising administering to the subject a pharmaceutical composition comprising one or more crystalline or amorphous forms of Bitopertin as described herein. These methods are particularly aimed at therapeutic and prophylactic treatments of animals, and more particularly, humans. The terms “subject,” an “individual,” or a “patient” are interchangeable throughout the specification and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In particular embodiments, the patient, subject or individual is a human.

The present application provides methods of preventing or treating erythropoietic protoporphyria (EPP), X-linked protoporphyria (XLPP), or congenital erythropoietic porphyria (CEP), or related syndrome (e.g., EPP-related syndrome, XLPP-related syndrome, or CEP-related syndrome) thereof in a subject, the method comprising administering to the subject one or more crystalline or amorphous forms of Bitopertin as described herein. The present application further provides methods of preventing or treating EPP, XLPP, or CEP in a subject, the method comprising administering to the subject one or more crystalline or amorphous forms of Bitopertin as described herein.

The present application further provides methods of preventing or treating EPP, XLPP, or CEP, or related syndrome thereof (e.g., EPP-related syndrome, XLPP-related syndrome, or CEP-related syndrome) in a subject, the method comprising administering to the subject a pharmaceutical composition comprising one or more crystalline or amorphous forms of Bitopertin as described herein. The present application further provides methods of preventing or treating EPP, XLPP, or CEP in a subject, the method comprising administering to the subject a pharmaceutical composition comprising one or more crystalline or amorphous forms of Bitopertin as described herein.

Erythropoietic protoporphyria (EPP) and X-linked protoporphyria (XLPP) are erythropoietic cutaneous porphyrias characterized by acute non-blistering photosensitivity, intolerance to sunlight, and significantly reduced quality of life. EPP is caused by a partial deficiency in ferrochelatase (FECH), which catalyzes the final step in the heme biosynthesis pathway. FECH deficiency increases levels of metal-free erythrocyte PPIX (also referred to herein as “free-protoporphyrin IX” and “PPIX”). XLPP is typically caused by C-terminal deletions in the ALAS2 gene which result in a gain-of-function mutation. These gain-of-function mutations increase the enzymatic activity of ALAS2 and cause an accumulation of both metal-free and zinc-bound PPIX. Both EPP and XLPP result in an accumulation of PPIX in erythrocytes and other tissues or biological fluids (e.g., skin, liver, bile, or stool). PPIX, which is lipophilic and eliminated via bile, is hepatotoxic at high concentrations.

Patients with EPP or XLPP usually develop photosensitivity during early childhood. Patients frequently present with symptoms of burning, itching, pain erythema, and edema on sun-exposed areas. Cutaneous symptoms are sometimes associated with abnormal liver enzyme activities, hepatobiliary injury, such as jaundice and liver cirrhosis, iron deficiency, and corresponding microcytic anemia.

The diagnosis of EPP and XLPP can be determined by measuring the levels of total erythrocyte, free-protoporphyrin IX, and zinc-protoporphyrin IX in hemolyzed anticoagulated whole blood. A diagnosis of EPP and/or XLPP can be made based on increased levels of free-protoporphyrin IX in blood. Patients with XLPP have a significantly higher proportion of zinc-protoporphyrin IX to free-protoporphyrin IX (e.g., >25%) as compared to those with EPP (e.g., ≤15%).

The diagnosis of EPP can also be determined by measuring the level of ferrocheletase activity in a subject. Ferrocheletase is a mitochondrial enzyme that catalyzes the insertion of ferrous iron into PPIX to form heme. Ferrocheletase also catalyzes the insertion of zinc, to form zinc protoporphyrin IX (ZPPIX) from any PPIX that remains after completion of heme synthesis. In EPP, free PPIX accumulates in bone marrow reticulocytes, since formation of both heme and ZPPIX is impaired. In some embodiments, the disclosure relates to methods of a treating a subject whose ferrochelatase activity level is reduced to between 10 to 35% of the ferrocheletase activity level observed in normal subjects. In some embodiments, the disclosure relates to methods of a treating a subject whose ferrochelatase activity level is reduced to less than 50% of the ferrocheletase activity level observed in normal subjects.

XLPP has a similar phenotype to EPP, and can be differentiated based on genetic analysis of ALAS2 or by determining the enzymatic activity level of ALAS2. In some embodiments, the disclosure relates to methods of a treating a subject having a gain-of-function mutation in ALAS2. In some embodiments, the subject's ALAS2 enzyme activity is increased. Since ferrocheletase is not deficient in XLPP, some of the excess PPIX measured in erythrocytes is ZPPIX and a lower percentage (e.g., 50-85%) is metal-free. In some embodiments, the subject has increased zinc-protoporphyrin IX levels in erythrocytes. In some embodiments, the method decreases zinc-protoporphyrin IX levels in the subject's erythrocytes. In some embodiments, method decreases zinc-protoporphyrin IX levels in the subject's erythrocytes by at least 10% (e.g., 10%, 15%, 20%, 25%, 30%, 35%, 40%, 45%, 50%, 55%, 60%, 65%, 70%, 75%, 80%, 85%, 90%, 95%, or at least 100%).

In certain aspects, the disclosure relates to methods of treating erythropoietic protoporphyria (EPP) and/or X-linked protoporphyria (XLPP) in a subject, the method comprising administering to the subject a pharmaceutical composition comprising one or more crystalline or amorphous forms of as described herein, wherein the subject has increased PPIX levels. In some embodiments, the method relates to subjects having PPIX levels that are at least 10%, 20%, 30%, 40%, or 50% more than PPIX levels in a healthy subject prior to administration of the crystalline or amorphous form(s) of Bitopertin as described herein. In some embodiments, the method relates to subjects having PPIX levels that are at least 10% more than PPIX levels in a healthy subject prior to administration of the crystalline or amorphous form(s) of Bitopertin. In some embodiments, the method relates to subjects having PPIX levels that are at least 20% more than PPIX levels in a healthy subject prior to administration of the crystalline or amorphous form(s) of Bitopertin. In some embodiments, the method relates to subjects having PPIX levels that are at least 30% more than PPIX levels in a healthy subject prior to administration of the crystalline or amorphous form(s) of Bitopertin. In some embodiments, the method relates to subjects having PPIX levels that are at least 40% more than PPIX levels in a healthy subject prior to administration of the crystalline or amorphous form(s) of Bitopertin. In some embodiments, the method relates to subjects having PPIX levels that are at least 50% more than PPIX levels in a healthy subject prior to administration of the crystalline or amorphous forms of Bitopertin. In some embodiments, the subject has increased protoporphyrin IX levels in the stool. In some embodiments, the subject has increased protoporphyrin IX levels in the skin. In some embodiments, the subject has increased free-protoporphyrin IX levels in erythrocytes. In some embodiments, the subject has greater than 31 μmol L−1 protoporphyrin IX levels in the erythrocytes. In some embodiments, the subject has between 31 μmol L−1 and 53 μmol L−1 protoporphyrin IX levels in the erythrocytes. In some embodiments, the subject has greater than 53 μmol L−1 protoporphyrin IX levels in the erythrocytes.

The present application further provides methods of inhibiting PPIX synthesis in vivo, comprising administering to a subject one or more crystalline or amorphous forms of Bitopertin as described herein. In certain aspects, the disclosure relates to methods of inhibiting PPIX synthesis in vivo, comprising administering to a subject one or more crystalline or amorphous forms of Bitopertin as described herein. In some embodiments, the disclosure relates to methods of inhibiting PPIX synthesis in vivo by at least 10% (e.g., 10%, 20%, 30%, 40%, 50%, 60%, 70%, 80%, 90%, or at least 100%). In some embodiments, the disclosure relates to methods of inhibiting PPIX synthesis in vivo by at least 20%. In some embodiments, the disclosure relates to methods of inhibiting PPIX synthesis in vivo by at least 30%. In some embodiments, the disclosure relates to methods of inhibiting PPIX synthesis in vivo by at least 40%. In some embodiments, the disclosure relates to methods of inhibiting PPIX synthesis in vivo by at least 50%. In some embodiments, the disclosure relates to methods of inhibiting PPIX synthesis in vivo by at least 60%. In some embodiments, the disclosure relates to methods of inhibiting PPIX synthesis in vivo by at least 70%. In some embodiments, the disclosure relates to methods of inhibiting PPIX synthesis in vivo by at least 80%. In some embodiments, the disclosure relates to methods of inhibiting PPIX synthesis in vivo by at least 90%. In some embodiments, the disclosure relates to methods of inhibiting PPIX synthesis in vivo by at least 100%. The present application further provides methods of decreasing the rate of PPIX synthesis in vivo, comprising administering to a subject one or more crystalline or amorphous forms of Bitopertin as described herein. In certain embodiments of the methods and uses as disclosed herein inhibit PPIX accumulation directly or indirectly. In certain such embodiments, PPIX accumulation is inhibited in a dose dependent manner. In certain embodiments of the foregoing methods, the glycine transporter inhibitor is a crystalline or amorphous form of Bitopertin as described herein. For example, the present application provides a method of inhibiting PPIX synthesis in vivo, decreasing the rate of PPIX synthesis in vitro, and/or inhibiting PPIX accumulation in vivo, comprising administering to a subject one or more crystalline or amorphous forms of as described herein.

In some embodiments, the method relates to methods of decreasing free-protoporphyrin IX levels in the subject. In some embodiments, the method relates to methods of decreasing free-protoporphyrin IX levels in the subject's erythrocytes. In some embodiments, the method decreases protoporphyrin IX levels in the erythrocytes of the subject to levels less than 53 μmol L⁻¹. In some embodiments, the method decreases protoporphyrin IX levels in the erythrocytes of the subject to levels less than 31 μmol L⁻¹. In some embodiments, the method decreases protoporphyrin IX levels in the erythrocytes of the subject to levels less than 15 μmol L⁻¹. In some embodiments, the method relates to decreasing protoporphyrin IX levels in the stool of the subject. In some embodiments, the method decreases protoporphyrin IX levels in the skin of the subject. In some embodiments, the method relates to methods of decreasing free-protoporphyrin IX levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method relates to methods of decreasing free-protoporphyrin IX levels in the subject by at least 15%. In some embodiments, the method relates to methods of decreasing free-protoporphyrin IX levels in the subject by at least 20%. In some embodiments, the method relates to methods of decreasing free-protoporphyrin IX levels in the subject by at least 25%. In some embodiments, the method relates to methods of decreasing free-protoporphyrin IX levels in the subject by at least 30%. In some embodiments, the method relates to methods of decreasing free-protoporphyrin IX levels in the subject by at least 35%. In some embodiments, the method relates to methods of decreasing free-protoporphyrin IX levels in the subject by at least 40%. In some embodiments, the method relates to methods of decreasing free-protoporphyrin IX levels in the subject by at least 45%. In some embodiments, the method relates to methods of decreasing free-protoporphyrin IX levels in the subject by at least 50%. In some embodiments, the method relates to methods of decreasing free-protoporphyrin IX levels in the subject by at least 55%. In some embodiments, the method relates to methods of decreasing free-protoporphyrin IX levels in the subject by at least 60%. In some embodiments, the method relates to methods of decreasing free-protoporphyrin IX levels in the subject by at least 65%. In some embodiments, the method relates to methods of decreasing free-protoporphyrin IX levels in the subject by at least 70%. In some embodiments, the method relates to methods of decreasing free-protoporphyrin IX levels in the subject by at least 75%. In some embodiments, the method relates to methods of decreasing free-protoporphyrin IX levels in the subject by at least 80%. In some embodiments, the method relates to methods of decreasing free-protoporphyrin IX levels in the subject by at least 85%. In some embodiments, the method relates to methods of decreasing free-protoporphyrin IX levels in the subject by at least 90%. In some embodiments, the method relates to methods of decreasing free-protoporphyrin IX levels in the subject by at least 95%. In some embodiments, the method relates to methods of decreasing free-protoporphyrin IX levels in the subject by at least 100%.

In certain aspects, the disclosure relates to methods of treating X-linked protoporphyria (XLPP) in a subject, the method comprising administering to the subject a pharmaceutical composition comprising one or more crystalline or amorphous forms of Bitopertin as described herein, wherein the subject has increased zinc-protoporphyrin IX (ZPPIX) levels. In some embodiments, the method relates to subjects having ZPPIX levels that are at least 10%, 20%, 30%, 40%, or 50% more than ZPPIX levels in a healthy subject prior to administration of the crystalline or amorphous form(s) of Bitopertin. In some embodiments, the method relates to subjects having ZPPIX levels that are at least 10% more than ZPPIX levels in a healthy subject prior to administration of the crystalline or amorphous form(s) of Bitopertin. In some embodiments, the method relates to subjects having ZPPIX levels that are at least 20% more than ZPPIX levels in a healthy subject prior to administration of the crystalline or amorphous form(s) of Bitopertin. In some embodiments, the method relates to subjects having ZPPIX levels that are at least 30% more than ZPPIX levels in a healthy subject prior to administration of the crystalline or amorphous form(s) of Bitopertin. In some embodiments, the method relates to subjects having ZPPIX levels that are at least 40% more than ZPPIX levels in a healthy subject prior to administration of the crystalline or amorphous form(s) of Bitopertin. In some embodiments, the method relates to subjects having ZPPIX levels that are at least 50% more than ZPPIX levels in a healthy subject prior to administration of the crystalline or amorphous form(s) of Bitopertin. In some embodiments, the subject has increased ZPPIX levels in erythrocytes.

In certain aspects, the disclosure relates to methods of treating X-linked protoporphyria (XLPP) in a subject, the method comprising administering to the subject a pharmaceutical composition comprising one or more the crystalline or amorphous form(s) of Bitopertin as described herein, wherein the subject has increased proportion of zinc-protoporphyrin IX (ZPPIX) to free-protoporphyrin IX (ZPPIX/PPIX ratio) as compared to those with EPP. In some embodiments, the method relates to subjects having a ZPPIX/PPIX ratio that is at least 15% (e.g., 15%, 20%, 25%, 30%, 35%, 40%, or 45%). In some embodiments, the method relates to subjects having a ZPPIX/PPIX ratio that is at least 20%. In some embodiments, the method relates to subjects having a ZPPIX/PPIX ratio that is at least 25%. In some embodiments, the method relates to subjects having a ZPPIX/PPIX ratio that is at least 30%. In some embodiments, the method relates to subjects having a ZPPIX/PPIX ratio that is at least 35%. In some embodiments, the method relates to subjects having a ZPPIX/PPIX ratio that is at least 40%. In some embodiments, the method relates to subjects having a ZPPIX/PPIX ratio that is at least 45%.

In certain aspects, the disclosure relates to methods of inhibiting zinc protoporphyrin IX (ZPPIX) synthesis in vivo, comprising administering to a subject one or more crystalline or amorphous form(s) of Bitopertin. In some embodiments, the disclosure relates to methods of inhibiting ZPPIX synthesis in vivo by at least 10% (e.g., 10%, 20%, 30%, 40%, 50%, 60%, 70%, 80%, 90%, or at least 100%). In some embodiments, the disclosure relates to methods of inhibiting ZPPIX synthesis in vivo by at least 20%. In some embodiments, the disclosure relates to methods of inhibiting ZPPIX synthesis in vivo by at least 30%. In some embodiments, the disclosure relates to methods of inhibiting ZPPIX synthesis in vivo by at least 40%. In some embodiments, the disclosure relates to methods of inhibiting ZPPIX synthesis in vivo by at least 50%. In some embodiments, the disclosure relates to methods of inhibiting ZPPIX synthesis in vivo by at least 60%. In some embodiments, the disclosure relates to methods of inhibiting ZPPIX synthesis in vivo by at least 70%. In some embodiments, the disclosure relates to methods of inhibiting ZPPIX synthesis in vivo by at least 80%. In some embodiments, the disclosure relates to methods of inhibiting ZPPIX synthesis in vivo by at least 90%. In some embodiments, the disclosure relates to methods of inhibiting ZPPIX synthesis in vivo by at least 100%.

In certain aspects, the disclosure relates to methods of treating erythropoietic protoporphyria (EPP), X-linked protoporphyria (XLPP), or congenital erythropoietic porphyria (CEP) in a subject, the method comprising administering to the subject a pharmaceutical composition comprising one or more the crystalline or amorphous form(s) of Bitopertin, wherein the subject has increased 5-aminolevulinic acid (5-ALA) levels. In some embodiments, the method relates to subjects having 5-ALA levels that are at least 10%, 20%, 30%, 40%, or 50% more than 5-ALA levels in a healthy subject prior to administration of the crystalline or amorphous form(s) of Bitopertin. In some embodiments, the method relates to subjects having 5-ALA levels that are at least 10% more than 5-ALA levels in a healthy subject prior to administration of the crystalline or amorphous form(s) of Bitopertin. In some embodiments, the method relates to subjects having 5-ALA levels that are at least 20% more than 5-ALA levels in a healthy subject prior to administration of the crystalline or amorphous form(s) of Bitopertin. In some embodiments, the method relates to subjects having 5-ALA levels that are at least 30% more than 5-ALA levels in a healthy subject prior to administration of the crystalline or amorphous form(s) of Bitopertin. In some embodiments, the method relates to subjects having 5-ALA levels that are at least 40% more than 5-ALA levels in a healthy subject prior to administration of the crystalline or amorphous form(s) of Bitopertin. In some embodiments, the method relates to subjects having 5-ALA levels that are at least 50% more than 5-ALA levels in a healthy subject prior to administration of the crystalline or amorphous form(s) of Bitopertin.

In certain aspects, the disclosure relates to methods of inhibiting 5-aminolevulinic acid (5-ALA) synthesis in vivo, comprising administering to a subject one or more crystalline or amorphous form(s) of Bitopertin. In some embodiments, the disclosure relates to methods of inhibiting 5-ALA synthesis in vivo by at least 10% (e.g., 10%, 20%, 30%, 40%, 50%, 60%, 70%, 80%, 90%, or at least 100%). In some embodiments, the disclosure relates to methods of inhibiting 5-ALA synthesis in vivo by at least 20%. In some embodiments, the disclosure relates to methods of inhibiting 5-ALA synthesis in vivo by at least 30%. In some embodiments, the disclosure relates to methods of inhibiting 5-ALA synthesis in vivo by at least 40%. In some embodiments, the disclosure relates to methods of inhibiting 5-ALA synthesis in vivo by at least 50%. In some embodiments, the disclosure relates to methods of inhibiting 5-ALA synthesis in vivo by at least 60%. In some embodiments, the disclosure relates to methods of inhibiting 5-ALA synthesis in vivo by at least 70%. In some embodiments, the disclosure relates to methods of inhibiting 5-ALA synthesis in vivo by at least 80%. In some embodiments, the disclosure relates to methods of inhibiting 5-ALA synthesis in vivo by at least 90%. In some embodiments, the disclosure relates to methods of inhibiting 5-ALA synthesis in vivo by at least 100%.

The present application further provides use of one or more crystalline or amorphous form(s) of Bitopertin as described herein in the manufacture of a formulation for the treatment of EPP, XLPP, CEP or related syndrome thereof (e.g., EPP-related syndrome, XLPP-related syndrome, or CEP-related syndrome) in a subject. In some embodiments, the present application provides use of one or more crystalline or amorphous form(s) of Bitopertin as described herein in the manufacture of a formulation for the treatment of EPP, XLPP, or CEP in a subject. In certain embodiments of the foregoing, the formulation is administered in a therapeutically effective amount.

The present application provides the use of one or more crystalline or amorphous form(s) of Bitopertin as described herein in the manufacture of a pharmaceutical composition for the treatment of EPP, XLPP, or CEP, or related syndrome thereof (e.g., EPP-related syndrome, XLPP-related syndrome, or CEP-related syndrome) in a subject. In some embodiments, the present application provides the use of one or more crystalline or amorphous form(s) of Bitopertin as described herein in the manufacture of a pharmaceutical composition for the treatment of EPP, XLPP, or CEP, in a subject. In certain embodiments of the foregoing, the pharmaceutical composition further comprises a pharmaceutically acceptable carrier.

Congenital erythropoietic porphyria (CEP) is an erythropoietic cutaneous porphyria characterized by blistering cutaneous photosensitivity. Severe cases of CEP can present in utero with hydrops fetalis, or shortly after birth with severe blistering photosensitivity, red urine, splenomegaly, hemolysis, and transfusion dependence. Milder cases and later onset forms typically present with red urine, severe blistering, and hemolytic anemia.

CEP individuals are often homozygous or compound heterozygous for UROS mutations. Some cases of CEP are due to mutations in the gene encoding the transcriptional regulator GATA1. These mutations result in reduced enzyme activity of uroporphyrinogen III synthase (UROIII-S), the fourth enzyme in the heme biosynthetic pathway. The decreased activity of UROIII-S leads to an accumulation of hydroxymethylbilane which spontaneously forms uroporphyrinogen I, which is further metabolized to coproporphyrinogen I. Uroporphyrinogen I and coproporphyrinogen I accumulate in the tissues.

The diagnosis of CEP can be determined by analyzing the enzyme activity of uroporphyrinogen III synthase (UROIII-S), by evaluating mutations in the UROS gene, by evaluating the function of GATA-1 erythroid-specific transcription factor, by evaluating mutations in GATA1, and by determining the levels of uroporphyrin I and coproporphyrin I in the subject. In some embodiments, the subject has a mutation in UROS. In some embodiments, the subject has a gene defect in GATA-1 erythroid-specific transcription factor. In some embodiments, the method relates to methods of treating a subject, wherein the subject has decreased activity of uroporphyrinogen III synthase. In some embodiments, the increased levels of uroporphyrin I and/or coproporphyrin I are measured in the subject's urine or red blood cells. In some embodiments, the increased levels of coproporphyrin I are measured in the subject's stool.

In certain aspects, the disclosure relates to methods of treating congenital erythropoietic porphyria (CEP) in a subject, the method comprising administering to the subject a pharmaceutical composition comprising one or more crystalline or amorphous form(s) of Bitopertin as described herein, wherein the subject has increased uroporphyrin I and/or coproporphyrin I levels. In some embodiments, the subject has increased levels of uroporphyrin I and/or coproporphyrin I. In some embodiments, the method relates to subjects having uroporphyrin I levels that are at least 10%, 20%, 30%, 40%, or 50% more than uroporphyrin I levels in a healthy subject prior to administration of the crystalline or amorphous form(s) of Bitopertin. In some embodiments, the method relates to subjects having uroporphyrin I levels that are at least 10% more than uroporphyrin I levels in a healthy subject prior to administration of the crystalline or amorphous form(s) of Bitopertin. In some embodiments, the method relates to subjects having uroporphyrin I levels that are at least 20% more than uroporphyrin I levels in a healthy subject prior to administration of the crystalline or amorphous form(s) of Bitopertin. In some embodiments, the method relates to subjects having uroporphyrin I levels that are at least 30% more than uroporphyrin I levels in a healthy subject prior to administration of the crystalline or amorphous form(s) of Bitopertin. In some embodiments, the method relates to subjects having uroporphyrin I levels that are at least 40% more than uroporphyrin I levels in a healthy subject prior to administration of the crystalline or amorphous form(s) of Bitopertin. In some embodiments, the method relates to subjects having uroporphyrin I levels that are at least 50% more than uroporphyrin I levels in a healthy subject prior to administration of the crystalline or amorphous form(s) of Bitopertin.

In some embodiments, the disclosure relates to methods of treating subjects having coproporphyrin I levels that are at least 10%, 20%, 30%, 40%, or 50% more than coproporphyrin I levels in a healthy subject prior to administration of the crystalline or amorphous form(s) of Bitopertin as described herein. In some embodiments, the method relates to subjects having coproporphyrin I levels that are at least 10% more than coproporphyrin I levels in a healthy subject prior to administration of the crystalline or amorphous form(s) of Bitopertin. In some embodiments, the method relates to subjects having coproporphyrin I levels that are at least 20% more than coproporphyrin I levels in a healthy subject prior to administration of the crystalline or amorphous form(s) of Bitopertin. In some embodiments, the method relates to subjects having coproporphyrin I levels that are at least 30% more than coproporphyrin I levels in a healthy subject prior to administration of the crystalline or amorphous form(s) of Bitopertin. In some embodiments, the method relates to subjects having coproporphyrin I levels that are at least 40% more than coproporphyrin I levels in a healthy subject prior to administration of the crystalline or amorphous form(s) of Bitopertin. In some embodiments, the method relates to subjects having coproporphyrin I levels that are at least 50% more than coproporphyrin I levels in a healthy subject prior to administration of the crystalline or amorphous form(s) of Bitopertin.

In certain aspects, the disclosure relates to methods of inhibiting uroporphyrin I and/or coproporphyrin I synthesis in vivo, comprising administering to a subject one or more crystalline or amorphous form(s) of Bitopertin as described herein. In some embodiments, the disclosure relates to methods of inhibiting uroporphyrin I synthesis in vivo by at least 10% (e.g., 10%, 20%, 30%, 40%, 50%, 60%, 70%, 80%, 90%, or at least 100%). In some embodiments, the disclosure relates to methods of inhibiting uroporphyrin I synthesis in vivo by at least 20%. In some embodiments, the disclosure relates to methods of inhibiting uroporphyrin I synthesis in vivo by at least 30%. In some embodiments, the disclosure relates to methods of inhibiting uroporphyrin I synthesis in vivo by at least 40%. In some embodiments, the disclosure relates to methods of inhibiting uroporphyrin I synthesis in vivo by at least 50%. In some embodiments, the disclosure relates to methods of inhibiting uroporphyrin I synthesis in vivo by at least 60%. In some embodiments, the disclosure relates to methods of inhibiting uroporphyrin I synthesis in vivo by at least 70%. In some embodiments, the disclosure relates to methods of inhibiting uroporphyrin I synthesis in vivo by at least 80%. In some embodiments, the disclosure relates to methods of inhibiting uroporphyrin I synthesis in vivo by at least 90%. In some embodiments, the disclosure relates to methods of inhibiting uroporphyrin I synthesis in vivo by at least 100%.

In some embodiments, the disclosure relates to methods of inhibiting coproporphyrin I synthesis in vivo by at least 10% (e.g., 10%, 20%, 30%, 40%, 50%, 60%, 70%, 80%, 90%, or at least 100%). In some embodiments, the disclosure relates to methods of inhibiting coproporphyrin I synthesis in vivo by at least 20%. In some embodiments, the disclosure relates to methods of inhibiting coproporphyrin I synthesis in vivo by at least 30%. In some embodiments, the disclosure relates to methods of inhibiting coproporphyrin I synthesis in vivo by at least 40%. In some embodiments, the disclosure relates to methods of inhibiting coproporphyrin I synthesis in vivo by at least 50%. In some embodiments, the disclosure relates to methods of inhibiting coproporphyrin I synthesis in vivo by at least 60%. In some embodiments, the disclosure relates to methods of inhibiting coproporphyrin I synthesis in vivo by at least 70%. In some embodiments, the disclosure relates to methods of inhibiting coproporphyrin I synthesis in vivo by at least 80%. In some embodiments, the disclosure relates to methods of inhibiting coproporphyrin I synthesis in vivo by at least 90%. In some embodiments, the disclosure relates to methods of inhibiting coproporphyrin I synthesis in vivo by at least 100%.

Porphyrins (e.g., PPIX, ZPPIX, uroporphyrin I, and coproporphyrin I) can be found in various biological samples including the skin, urine, stool, plasma, and erythrocytes. In some embodiments, the porphyrins may be extracted from the biological sample into a solution for fluorescence analysis. Porphyrins can be detected in these biological samples by direct inspection using long wavelength ultraviolet light (e.g., 400-420 nm light). Porphyrins have the greatest absorption wavelengths near 400-420 nm, with their highest absorption peak occurring at 415 nm. The emission maxima of porphyrins is typically around 600 nm and varies slightly based on the type of porphyrins and the solvent used for analysis. In some embodiments, diagnosis of EPP, XLPP, and CEP may be made using fluorescence analysis. In some embodiments, skin porphyrin levels (e.g., PPIX levels) can be measured by calculating the difference before and after complete photobleaching of PPIX using controlled illumination. See, e.g., Heerfordt IM. Br J Dermatol. 2016; 175 (6): 1284-1289.

In some embodiments, the subject's plasma porphyrin fluoresces at a peak of 634 nm when illuminated with blue light (e.g., 400-420 nm light). In some embodiments, the subject's plasma porphyrin fluoresces at a peak between 626 nm and 634 nm when illuminated with blue light (e.g., 400-420 nm light). In some embodiments, the subject's skin porphyrin fluoresces at a peak of 632 nm when illuminated with blue light (e.g., 400-420 nm light). In some embodiments, the subject's skin porphyrin fluoresces at a peak between 626 nm and 634 nm when illuminated with blue light (e.g., 400-420 nm light). In some embodiments, the subject has greater than 0.2 FluoDerm Units (FDU) of protoporphyrin IX levels in the skin. In some embodiments, the subject has greater than 1.0 FDU of protoporphyrin IX levels in the skin. In some embodiments, the subject has between 1.0 FDU and 2.5 FDU of protoporphyrin IX levels in the skin. In some embodiments, the subject has greater than 2.5 FDU of protoporphyrin IX levels in the skin. In some embodiments, the method decreases protoporphyrin IX levels in the skin of the subject to less than 0.5 FDU. In some embodiments, the method decreases protoporphyrin IX levels in the skin of the subject to less than 1.0 FDU. In some embodiments, the method decreases protoporphyrin IX levels in the skin of the subject to less than 1.5 FDU. In some embodiments, the method decreases protoporphyrin IX levels in the skin of the subject to less than 2.0 FDU. In some embodiments, the method decreases protoporphyrin IX levels in the skin of the subject to less than 2.5 FDU. In some embodiments, the subject has red fluorescent urine. In some embodiments, the subject has a peak between 615 nm and 620 nm using plasma porphyrin fluorescence analysis.

In certain aspects, the disclosure relates to methods of preventing, treating, or reducing the progression rate and/or severity of one or more complications of EPP, XLPP, or CEP in a subject, the method comprising administering to the subject a pharmaceutical composition comprising one or more crystalline or amorphous form(s) of Bitopertin described herein. In some embodiments, the one or more complications of EPP, XLPP, or CEP is selected from the group consisting of: acute photosensitivity, cutaneous photosensitivity, edema, erythema, anemia, hypochromic anemia, hemolytic anemia, hemolysis, mild hemolysis, severe hemolysis, chronic hemolysis, hypersplenism, palmar keratoderma, bullae, lesions, scarring, deformities, loss of fingernails, loss of digits, cholelithiasis, cholestasis, cytolysis, gallstones, cholestatic liver failure, erythrodontia, hypercellular bone marrow, myelodysplasia, thrombocytopenia, hydrops fetalis and/or death in utero. In some embodiments, the disclosure contemplates methods of treating one or more complications of EPP, XLPP, or CEP (e.g., acute photosensitivity, cutaneous photosensitivity, edema, erythema, anemia, hypochromic anemia, hemolytic anemia, hemolysis, mild hemolysis, severe hemolysis, chronic hemolysis, hypersplenism, palmar keratoderma, bullae, lesions, scarring, deformities, loss of fingernails, loss of digits, cholelithiasis, cholestasis, cytolysis, gallstones, cholestatic liver failure, erythrodontia, hypercellular bone marrow, myelodysplasia, thrombocytopenia, hydrops fetalis and/or death in utero) comprising administering to the subject a pharmaceutical composition comprising one or more crystalline or amorphous form(s) of Bitopertin as described herein. In some embodiments, the one or more complications are improved indirectly. In some embodiments, the disclosure contemplates methods of preventing one or more complications of EPP, XLPP, or CEP comprising administering to the subject a pharmaceutical composition comprising one or more crystalline or amorphous form(s) of Bitopertin as described herein. In some embodiments, the disclosure contemplates methods of reducing the progression rate of one or more complications of EPP, XLPP, or CEP comprising administering to the subject a pharmaceutical composition comprising one or more crystalline or amorphous form(s) of Bitopertin. In some embodiments, the disclosure contemplates methods of reducing the severity of one or more complications of EPP, XLPP, or CEP comprising administering to the subject a pharmaceutical composition comprising one or more crystalline or amorphous form(s) of Bitopertin as described herein.

Optionally, methods disclosed herein for preventing, treating, or reducing the progression rate and/or severity of one or more complications of EPP, XLPP, or CEP in a subject, may further comprise administering to the patient one or more supportive therapies or additional active agents for treating EPP, XLPP, or CEP. For example, the patient also may be administered one or more supportive therapies or active agents selected from the group consisting of: avoiding sunlight, topical sunscreens, skin protection, UVB phototherapy, Afamelanotide (Scenesse®), bortezomib, proteasome inhibitors, chemical chaperones, cholestyramine, activated charcoal, iron supplementation, liver transplantation, bone marrow transplantation, splenectomy, and blood transfusion. In some embodiments, the methods described herein may further comprise administering to the patient Afamelanotide (Scenesse®).

Porphyrin photosensitization in EPP, XLPP, and CEP produces two distinct clinical syndromes: (1) acute photosensitivity on exposure to sunlight with erythema and edema and (2) a syndrome wherein subepidermal bullae occur in sun-exposed areas of the skin. In certain aspects, the disclosure relates to methods of preventing, treating, or reducing the progression rate and/or severity of EPP, XLPP, or CEP in a subject, the method comprising administering to the subject a pharmaceutical composition comprising one or more crystalline or amorphous form(s) of Bitopertin as described herein, wherein the method increases pain free light exposure in the subject. In some embodiments, the method increases pain free light exposure in the subject by at least 10%, 20%, 30%, 40%, or 50% more as compared to pain free light exposure prior to administration of the crystalline or amorphous form(s) of Bitopertin. In some embodiments, the method decreases light sensitivity in the subject. In some embodiments, the method decreases light sensitivity in the subject by at least 10%, 20%, 30%, 40%, or 50% more as compared to light sensitivity prior to administration of the crystalline or amorphous form(s) of Bitopertin. In some embodiments, the subject has a history of phototoxic reactions from EPP. In some embodiments, the subject is an adult, child, infant, or pregnant woman.

Glycine is one of the key initial substrates for heme and globin synthesis. As such, decreased levels of glycine due to GlyT1 inhibition could lead to a decrease in heme synthesis. In certain aspects, the disclosure relates to methods of treating EPP, XLPP, or

CEP in a subject, the method comprising administering to the subject a pharmaceutical composition comprising one or more crystalline or amorphous form(s) of Bitopertin as described herein, wherein the subject's heme levels decrease no more than 10% (e.g., 10%, 15%, 20%, 25%, and 30%). In some embodiments, the disclosure relates to methods of treating EPP, XLPP, or CEP in a subject, wherein the subject's heme levels decrease no more than 15%. In some embodiments, the disclosure relates to methods of treating EPP, XLPP, or CEP in a subject, wherein the subject's heme levels decrease no more than 20%. In some embodiments, the disclosure relates to methods of treating EPP, XLPP, or CEP in a subject, wherein the subject's heme levels decrease no more than 25%. In some embodiments, the disclosure relates to methods of treating EPP, XLPP, or CEP in a subject, wherein the subject's heme levels decrease no more than 30%.

In certain aspects, the disclosure relates to methods of treating EPP, XLPP, or CEP in a subject, the method comprising administering to the subject a pharmaceutical composition comprising one or more crystalline or amorphous form(s) of Bitopertin, wherein the subject's PPIX levels decrease while the patient's heme levels are substantially maintained. In some embodiments, the patients PPIX levels decrease by at least 50% (e.g., 50%, 55%, 60%, 65%, 70%, 75%, 80%, 85%, 90%, 95%, or at least 100%) and the patient's heme levels decrease no more than 10% (e.g., 10%, 15%, 20%, 25%, and 30%). In some embodiments, the patient's PPIX levels decrease by at least 85% and the patient's heme levels decrease no more than 15%. In some embodiments, the patients PPIX levels decrease by at least 80% and the patient's heme levels decrease no more than 15%. In some embodiments, the patients PPIX levels decrease by at least 75% and the patient's heme levels decrease no more than 15%. In some embodiments, the patients PPIX levels decrease by at least 70% and the patient's heme levels decrease no more than 15%. In some embodiments, the patients PPIX levels decrease by at least 65% and the patient's heme levels decrease no more than 15%. In some embodiments, the patients PPIX levels decrease by at least 60% and the patient's heme levels decrease no more than 15%. In some embodiments, the patients PPIX levels decrease by at least 55% and the patient's heme levels decrease no more than 15%. In some embodiments, the patients PPIX levels decrease by at least 50% and the patient's heme levels decrease no more than 15%.

In certain aspects, the disclosure relates to methods of treating EPP, XLPP, or CEP in a subject, the method comprising administering to the subject a pharmaceutical composition comprising one or more crystalline or amorphous form(s) of Bitopertin, wherein the dosage of the pharmaceutical composition does not cause a substantial reduction in heme levels. In some embodiments, the patient's PPIX levels decrease by at least 50% (e.g., 50%, 55%, 60%, 65%, 70%, 75%, 80%, 85%, 90%, 95%, or at least 100%). In some embodiments, the patient's PPIX levels decrease by at least 55%. In some embodiments, the patient's PPIX levels decrease by at least 60%. In some embodiments, the patient's PPIX levels decrease by at least 65%. In some embodiments, the patient's PPIX levels decrease by at least 70%. In some embodiments, the patient's PPIX levels decrease by at least 75%. In some embodiments, the patient's PPIX levels decrease by at least 80%. In some embodiments, the patient's PPIX levels decrease by at least 85%. In some embodiments, the patient's PPIX levels decrease by at least 90%. In some embodiments, the patient's PPIX levels decrease by at least 95%. In some embodiments, the patient's PPIX levels decrease by at least 100%. In some embodiments, the patient's heme levels decrease no more than 10% (e.g., 10%, 15%, 20%, 25%, and 30%). In some embodiments, the patient's heme levels decrease no more than 15%. In some embodiments, the patient's heme levels decrease no more than 20%. In some embodiments, the patient's heme levels decrease no more than 25%. In some embodiments, the patient's heme levels decrease no more than 30%.

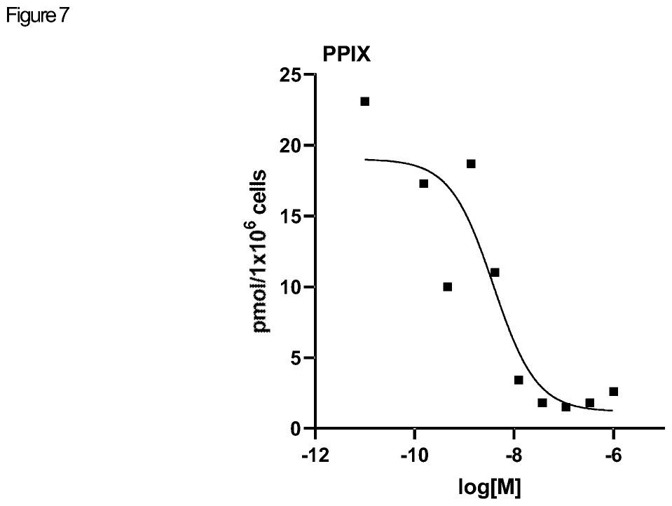

In some embodiments, the accumulation of one or more of the following heme intermediates is inhibited, wherein the one or more heme intermediates is selected from the group consisting of PPIX, ZPPIX, uroporphyrin I, coproporphyrin I, and/or 5-ALA. In some embodiments, the disclosure relates to methods of inhibiting the accumulation of PPIX, the method comprising administering to the subject a pharmaceutical composition comprising one or more crystalline or amorphous form(s) of Bitopertin as described herein. In some embodiments, the disclosure relates to methods of inhibiting the accumulation of ZPPIX, the method comprising administering to the subject a pharmaceutical composition comprising one or more crystalline or amorphous form(s) of Bitopertin as described herein. In some embodiments, the disclosure relates to methods of inhibiting the accumulation of uroporphyrin I, the method comprising administering to the subject a pharmaceutical composition comprising one or more crystalline or amorphous form(s) of Bitopertin as described herein. In some embodiments, the disclosure relates to methods of inhibiting the accumulation of coproporphyrin I, the method comprising administering to the subject a pharmaceutical composition comprising one or more crystalline or amorphous form(s) of Bitopertin as described herein. In some embodiments, the disclosure relates to methods of inhibiting the accumulation of 5-ALA, the method comprising administering to the subject a pharmaceutical composition comprising one or more crystalline or amorphous form(s) of Bitopertin as described herein. In some embodiments, the accumulation of the one or more heme intermediates (e.g., PPIX, ZPPIX, uroporphyrin I, coproporphyrin I, and/or 5-ALA) is inhibited in a dose dependent manner. See, e.g., FIG. 7.

Protoporphyrin accumulation in EPP, XLPP, and CEP can cause liver damage when the hepatic load exceeds the canalicular excretion capacity. The accumulation of PPIX in hepatocytes and bile canaliculi may result in cell damage, cholestasis, cytolysis and further retention of protoporphyrin. Excess protoporphyrin can exert cholestatic effects leading to changes in the hepatobiliary system which can range from mild inflammation to fibrosis and cirrhosis (e.g., cholelithiasis, mild liver disease, deteriorating liver disease, and terminal phase liver disease). Between 3-5% of patients with EPP or XLPP develop protoporphyric hepatopathy, a severe liver disease that can progress rapidly and require liver transplantation. Approximately 2% of patients will develop severe liver disease.

In certain aspects, the disclosure relates to methods of preventing, treating, or reducing the progression rate and/or severity of liver disease associated with EPP, XLPP, or CEP in a subject, the method comprising administering to the subject a pharmaceutical composition comprising one or more crystalline or amorphous form(s) of Bitopertin as described herein. In some embodiments, the liver disease associated with EPP, XLPP, or

CEP is cholelithiasis. In some embodiments, the liver disease associated with EPP, XLPP, or CEP is mild liver disease. In some embodiments, the liver disease associated with EPP, XLPP, or CEP is deteriorating liver disease. In some embodiments, the liver disease associated with EPP, XLPP, or CEP is terminal phase liver disease.

Liver function in patients with EPP, XLPP, and CEP can be assessed using various known clinical assays. In some embodiments, liver function tests can be used to determine the level of various biochemical parameters (e.g., raised aspartate transaminase levels, alkaline phosphatase, or γ-glutamyl transferase levels). In some embodiments, histopathology of liver biopsies may be used to assess one or more parameters (e.g., protoporphyrin deposition, fibrosis, infiltrates, portal fibrosis, and periportal fibrosis) in the subject. In some embodiments, ultrastructural studies of biopsy specimen can be used to determine if crystal containing vacuoles are present in the subject. With deterioration of liver function, urinary coproporphyrin excretion increases. In some embodiments, coproporphyrin excretion in the urine may be analyzed to assess liver function in the subject. In some embodiments, ultrasound or magnetic resonance elastography may be used to measure liver stiffness in the subject.

In certain embodiments of the methods and uses as disclosed herein, the crystalline or amorphous form of Bitopertin as described herein demonstrates PPIX inhibition with an EC50 of less than 500 nM, less than 400 nM, less than 300 nM, less than 200 nM, or less than 100 nM. In certain embodiments of the present application, the crystalline or amorphous form of Bitopertin demonstrates PPIX inhibition with an EC50 of less than 100 nM. In certain embodiments of the present application, the crystalline or amorphous form of Bitopertin demonstrates PPIX inhibition with an EC50 of less than 50 nM. In certain such embodiments, the EC50 is measured in a flow cytometry assay. In certain embodiments of the foregoing, the GlyT1 inhibitor is Bitopertin or a solid form thereof (e.g., a crystalline or an amorphous form descried herein).

In certain embodiments of the methods and uses as disclosed herein, at least 50%, at least 60%, at least 70%, at least 80%, at least 85%, at least 90%, or at least 95% cell viability is maintained. In certain such embodiments, at least 90% cell viability is maintained.

The present disclosure also provides the following non-limiting embodiments:

In order that the embodiments disclosed herein may be more efficiently understood, examples are provided below. It should be understood that these examples are for illustrative purposes only and are not to be construed as limiting the embodiments in any manner.

Throughout these examples, there may be molecular cloning reactions, and other standard recombinant DNA techniques described and these were carried out according to methods described in Maniatis et al., Molecular Cloning—A Laboratory Manual, 2nd ed., Cold Spring Harbor Press (1989), using commercially available reagents, except where otherwise noted.

The following examples are illustrative, but not limiting, of the methods and compositions described herein. Other suitable modifications and adaptations of the variety of conditions and parameters normally encountered in therapy, synthesis, and other embodiments disclosed herein are within the spirit and scope of the embodiments.

### EXAMPLES

### Example 1: Synthesis of Compounds

The compounds disclosed herein can be made in accordance with well-known procedures and by processes known and disclosed in the art. For example, Bitopertin, can be prepared in accordance with the synthetic protocols provided in U.S. Pat. Nos. 7,319,099, 9,877,963, and 7,812,161, the contents of which are hereby incorporated by reference in their entirety.

### Example 2: GlyT1 Inhibitors to Treat Subjects with Erythropoietic Protoporphyrias (EPP), X-Linked Protoporphyria (XLPP), and Congenital Erythropoietic Porphyria (CEP). Prophetic Example

The synthesis of large amounts of heme is a fundamental requirement in the developing erythroid cell in order to support the production of large amounts of hemoglobin. In this cell lineage, the amount of heme needed to meet this demand is greatly in excess of any other cell type. Heme synthesis is initiated with the condensation of glycine with succinyl CoA by the enzyme ALAS. This is the rate-limiting step in heme biosynthesis to ensure that heme intermediates do not accumulate and cause toxicity. Erythroid cells have acquired an erythroid specific form of ALAS (ALAS2) and the glycine transporter GlyT1 to increase glycine availability in order to meet this high demand for heme.

Animal and human studies in which the activity of GlyT1 is eliminated by gene deletion (Garcia-Santos et al, 2017) or decreased by administration of a specific GlyT1 inhibitor (Pinard et al, 2018) have been shown to reduce heme synthesis in erythroid cells, leading to moderate microcytic hypochromic anemia as a consequence of impaired hemoglobin production. These findings indicate that modulation of glycine uptake in red cells is able to regulate the heme biosynthetic pathway.

In patients with either the erythropoietic protoporphyrias or congenital erythropoietic porphyria specific mutations in individual genes encoding enzymes of the heme biosynthetic pathway lead to altered enzyme activity and the accumulations of heme intermediates upstream of the affected enzyme. Accumulation of these metabolites occurs because the mutated enzyme becomes the rate-limiting step in the pathway with activity that is insufficient to fully convert the upstream metabolite to the next step in the pathway. Three diseases are of specific interest:

1. EPP caused by mutations in the ferrochetalase gene that leads to reduced activity of this enzyme and accumulation of the upstream metabolite protoporphyrin IX (PPIX). Rarely EPP may be observed in an acquired form in older humans who have developed a novel clone containing a ferrochetalase mutation as a feature of myelodysplasia

2. XLPP caused by activating mutations in the ALAS2 gene, leading to high levels of PPIX. In this case, the accumulating metabolite is downstream of the affected enzyme because of overproduction that cannot be fully converted to heme even by normal levels of ferrochetalase.

3. CEP caused by mutations in the gene for uroporphyrinogen synthase resulting in reduced activity of this enzyme and accumulation of the upstream metabolite coproporphyrin I.

These heme intermediates may escape the red cell either by hemolysis of the cell (in CEP) or by active transport out of the cell (in EPP and XLPP) and cause toxicity. A consistent feature of all three diseases is a severe, painful, blistering skin reactions following exposure to sunlight that causes permanent scarring and deformity. This is caused by the local production of reactive intermediates by the action of sunlight on PPIX or coproporphyrin I that provokes a severe inflammatory reaction. PPIX is hydrophobic and therefore excreted through the biliary tract. High biliary concentrations may lead to cholelithiasis, cholestasis, and hepatic damage which can be severe, leading to hepatic failure. In the case of CEP, accumulation of coproporphyrin within the mature red cell may lead to severe hemolytic anemia.

These disease manifestations of EPP, XLPP and CEP are caused by overproduction of intermediate heme metabolites due to genetic abnormalities in the heme biosynthetic pathway. The accumulated metabolites are either toxic to the red cell, following accumulation in the skin and exposure to sunlight, or because of biliary excretion by the liver. GlyT1 controls the availability of one of the initial substrates in the heme biosynthetic pathway and has been shown to downregulate heme production in humans or animals with a normal heme pathway as described herein. Without being bound to any particular theory, GlyT1 is able to reduce the production of intermediate metabolites of heme in the same way, particularly when those intermediate products accumulate as a result of abnormal enzyme activity. Therefore, a subject with EPP, XLPP or CEP is treated with a GlyT1, which will reduce of the production of toxic metabolites in erythroid cells in such subjects and leads to a reduction in skin accumulation of these metabolites, reduced hepatobiliary excretion, or in the case of CEP a reduction in hemolysis, in all cases a reduction in disease severity. Thus, the disease is treated.

### Example 3: Met GlyT1 Inhibitors Are Effective to Reduce the Level of Heme Metabolites in Erythroleukemia Cell Lines Containing Disease-Causing Mutations for EPP, XLPP or CEP

Erythroleukemia cells are genetically modified to obtain cell lines containing disease-causing mutations for EPP, XLPP or CEP. These genetically modified cell lines are treated with GlyT1 inhibitors and the production of heme metabolites is evaluated photometrically, biochemically or in radiolabel studies. The level of photohemolysis caused by PPIX is assessed in these cell lines and is found to be reduced in the presence of GlyT1 inhibitors.

### Example 4: GlyT1 Inhibitors are Effective to Reduce the Level of Heme Metabolites in Erythroid Cells Containing Disease-Causing Mutations for EPP, XLPP or CEP Prophetic Example

Erythroid cells are taken from bone marrow or peripheral blood of animals with disease causing mutations in the specific genes responsible for EPP, XLPP or CEP. These cell lines are treated with GlyT1 inhibitors and the production of heme metabolites is evaluated photometrically, biochemically or in radiolabel studies. The level of photohemolysis caused by PPIX is assessed in these cell lines and is found to be reduced in the presence of GlyT1 inhibitors.

### Example 5: GlyT1 Inhibitors are Effective to Reduce the Level of Heme Metabolites in Patients' Erythroid Cells Containing Disease-Causing Mutations for EPP, XLPP or CEP. Prophetic Example

Erythroid cells (reticulocytes and erythrocytes) are obtained from patients with EPP, XLPP and CEP (as available). These cells from patients are treated with GlyT1 inhibitors and the production of heme metabolites is evaluated photometrically, biochemically or in radiolabel studies. The level of photohemolysis caused by PPIX is assessed in these cell lines and is found to be reduced in the presence of GlyT1 inhibitors.

### Example 6: GlyT1 Inhibitors Are Effective to Reduce the Severity of EPP or XLPP in Animals. (Prophetic Example

Animals with EPP and XLPP are treated over a period with one or more GlyT1 inhibitors at various doses. The level of toxic heme intermediates in such animals is found to be reduced and the symptoms of such diseases, such as the severity of cutaneous reactions, hepatobiliary disease and/or hemolysis are found to be ameliorated.

The embodiments and examples provided herein demonstrate that the GlyT1 inhibitors can be used to treat EPP, XLPP, or CEP. This is a surprising and unexpected result.

### Example 7: EPP Cellular Model

Knockout guide sequences were designed to target Exon3 of the Ferrochelatase gene. Guide sequences tested are shown in Table 5.

TABLE 5
Guide sequences tested

| | | | |
|---|---|---|---|
| FECH_gRNA_3_KO | ATGGGAGGCCCTGA | gRNA | Exon3 |
| | AACTCT | | |
| | (SEQ ID NO: 1) | | |
| FECH_gRNA_4_KO | TGAAACTCTTGGAG | gRNA | Exon3 |
| | ATGTTC | | |
| | (SEQ ID NO: 2) | | |
| FECH_gRNA_5_KO | TCTGAGACTCTTCT | gRNA | Exon3 |
| | TGGACC | | |
| | (SEQ ID NO: 3) | | |

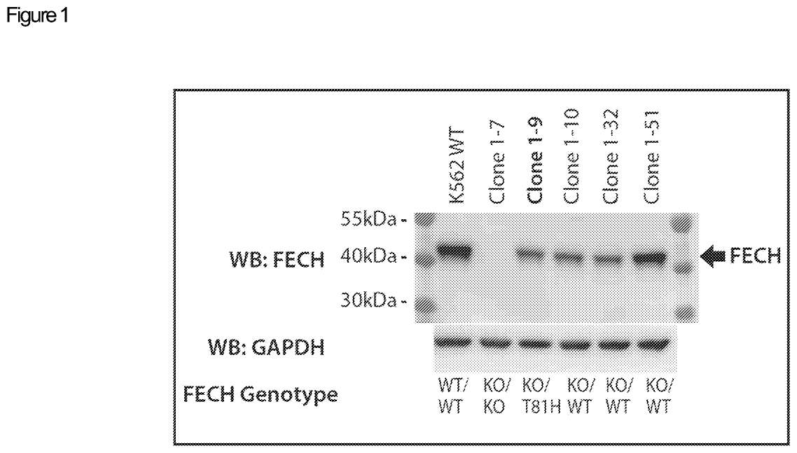

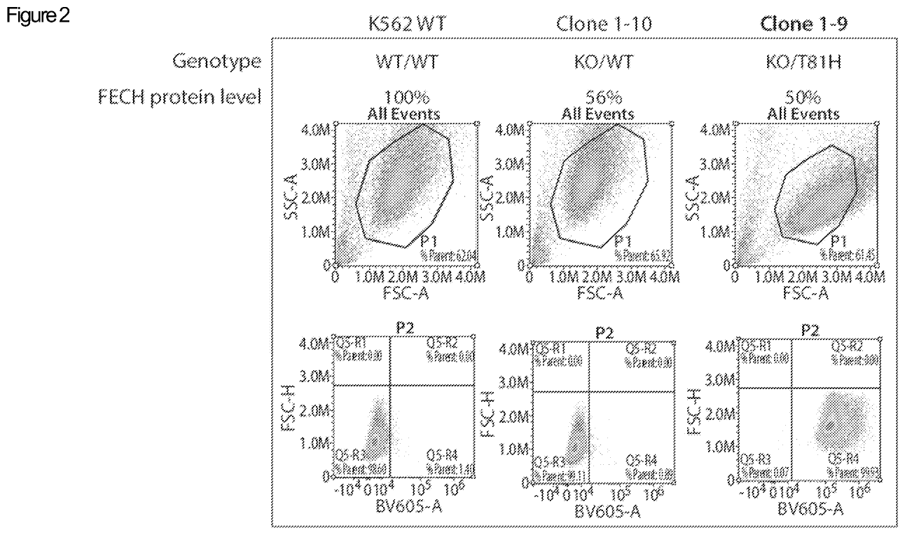

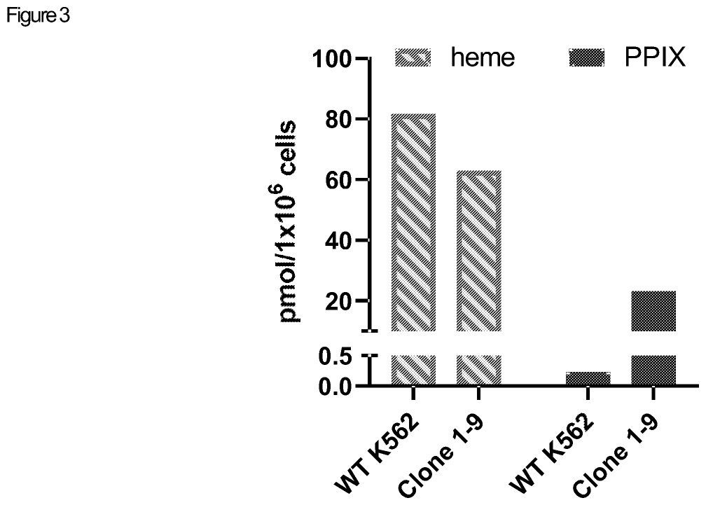

K562 cells were cultured in Iscove's Modified Dulbecco's Medium (IMDM) supplemented with 10% fetal bovine serum (FBS), 1% penicillin/streptomycin (PS). CRISPR Cas9 RNP complex with guide RNA was electroporated in K562 cells. Genomic DNA from the pooled cells was isolated, amplified by PCR and sequenced by Sanger sequencing to determine knockout efficiency. Single-cell clones were isolated by fluorescent-assisted cell sorting (FACS). TA cloning and Sanger sequencing were used to confirm single-cell clones and genotypes. Five clones were selected (Clone IDs are: Clone 1-7; Clone 1-9; Clone 1-10, Clone 1-32; Clone 1-51; and K562 WT) for further characterization by western blot (FIG. 1) to determine FECH protein expression levels (Antibody: FECH Antibody Rabbit Polyclonal, Proteintech, 14466-1-AP) and flow cytometry to determine the PPIX levels (FIG. 2). LC/MS/MS confirmed the accumulation of PPIX in clone 1-9 compared to WT K562 cells (FIG. 3). The genotypic characteristics of the five clones is as provided in Table 6.

TABLE 6
Genotypic Characteristics of Clones

| Clone ID | Genotype |
|---|---|
| K562 WT | WT/WT |
| 1-7 | KO/KO |
| 1-9 | KO/Missense T81H |
| 1-10 | KO/WT |
| 1-32 | KO/WT |
| 1-51 | KO/WT |

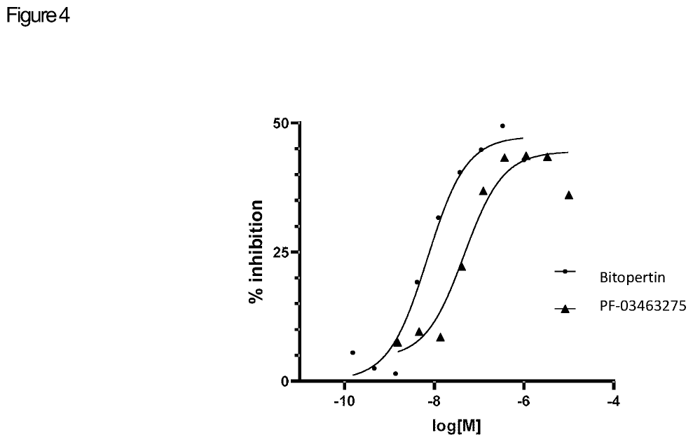

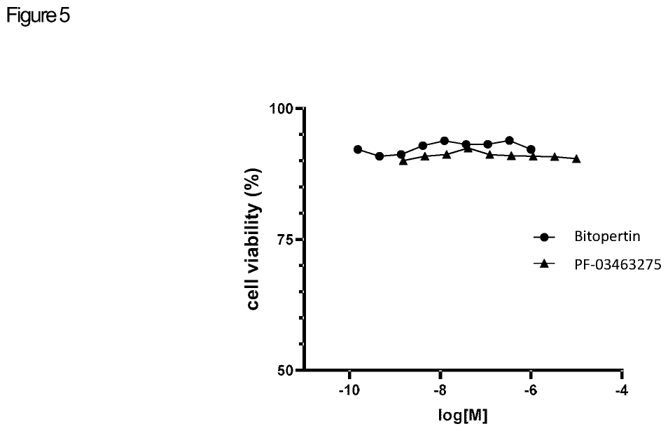

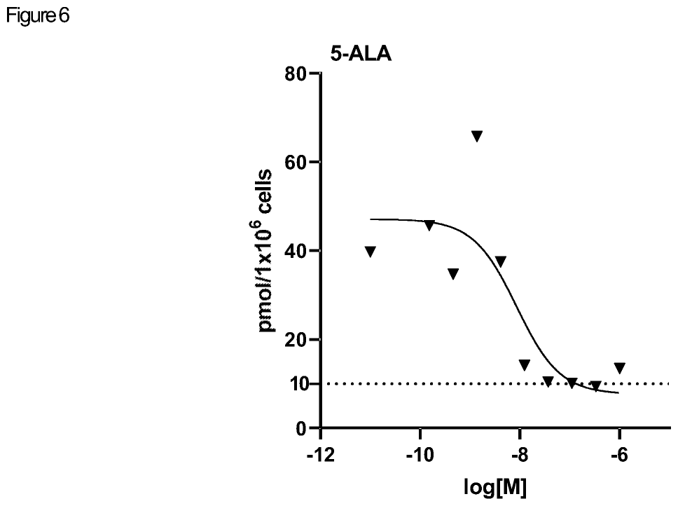

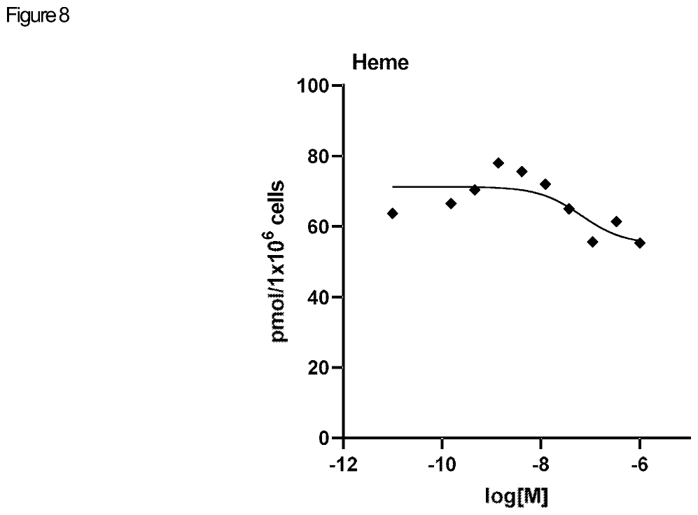

900 μL of K562 clone-9 cells at 2×10⁵ cells/mL in IMDM media with 10% FBS and 1% PS were plated into a 24-well plate. After 24-hour incubation, 100 μL of compound in DMSO/media was added in different concentration. The final concentration of DMSO was 0.1%. The compound was incubated for 96 hours at 37° C. Cell viability and cell count were measured by Vi-CELL XR Complete System. Finally, the effect of the compound on PPIX level was determined by flow cytometry. FIG. 4 shows that both Bitopertin and PF-03463275 demonstrated a dose-dependent inhibition of PPIX accumulation by flow cytometry, up to 50%. Bitopertin exhibited an EC50 of 7 nM and PF-03463275 exhibited an EC50 of 46 nM FIG. 5 shows that both Bitopertin and PF-03463275 had no negative effects on cell viability. Importantly, LC/MS/MS method demonstrated that Bitopertin decreased 5-aminolevulinic acid (5-ALA) and PPIX levels in the EPP K562 cellular model with minimal effect on Heme formation (FIGS. 6, 7, and 8).

Additional GlyT1 inhibitors also showed a dose-dependent inhibition of PPIX accumulation, while ORG-25543,

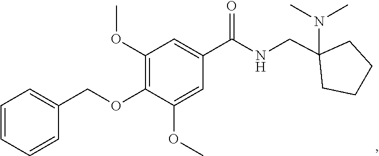 [SMILES: COc1cc(C(=O)NCC2(N(C)C)CCCC2)cc(OC)c1OCc1ccccc1]

a GlyT2 inhibitor, did not show any inhibition up to the highest tested concentration of 10 μM (Table 7).

TABLE 7
EC₅₀ of tested compounds in the EPP cellular model

| | EPP cellular model |
|---|---|
| Compound name | (EC₅₀ nM) |
| Bitopertin | 7.0 |
| PF-03463275 | 46 |
| SSR-504734 | 306 |
| ALX-5407 | 0.34 |
| ORG-25935 | 14 |
| ORG-24598 | 5.6 |
| LY-2365109 | 4.1 |
| ORG-25543 | >10,000 |

### Example 8: GlyT1 Inhibitors are Effective to Reduce PPIX Level in Human Hematopoietic Stem Cells Transduced with Lentiviruses Expressing Small Interference RNA (shRNA) of FECH

To investigate the effects of GlyT1 inhibitors in human hematopoietic stem cells with an EPP phenotype, lentiviral vectors expressing shRNA of FECH were constructed (Table 4) and transduced at 25 MOI into human cord blood-CD34+ cells purchased from Stemexpress.

TABLE 4
Oligos used to construct lentiviral
vectors with shRNA sequences of FECH

| | |
|---|---|
| FECH- | CCGGGCTTTGCAGATCATATTCTAACTCGAGTTAGAA |
| shRNA1- | TATGATCTGCAAAGCTTTTTG (SEQ ID NO: 4) |
| F | |
| FECH- | AATTCAAAAAGCTTTGCAGATCATATTCTAACTCGAG |
| shRNA1- | TTAGAATATGATCTGCAAAGC (SEQ ID NO: 5) |
| R | |
| Plasmid | pLKO.1 TRC cloning vector |

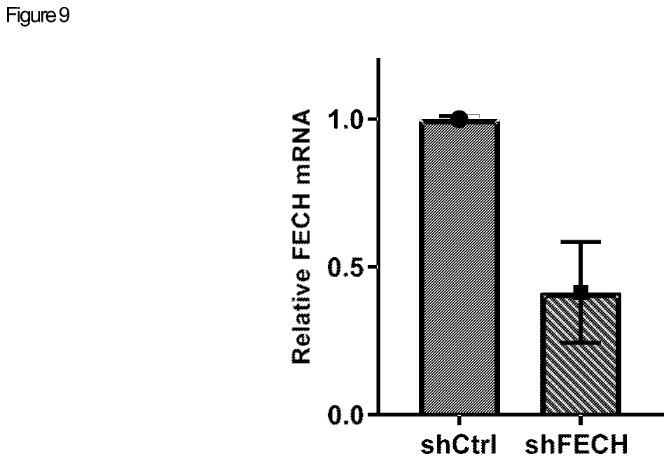

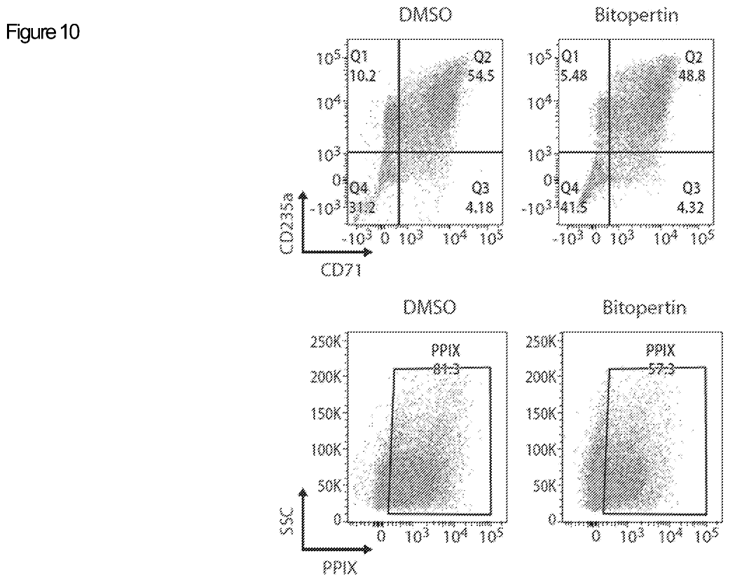

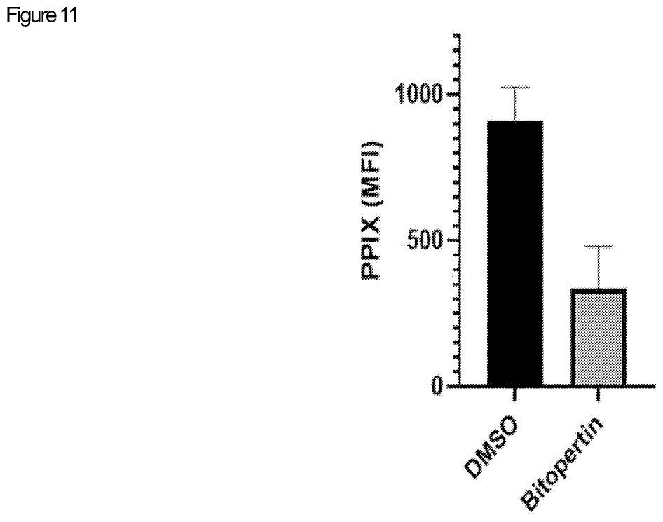

RT-qPCR of the resulted CD34+ cells shows 60% reduction in FECH mRNA level relative to that of cells treated with control lentiviral vectors (FIG. 9). Transduced CD34+ cells were differentiated in the presence of Bitopertin (100 nM) or DMSO control into erythroid cells for 9 days in StemSpan SFEM II media supplemented with StemSpan Erythroid Expansion Supplement. The erythroid cell antigen profile was analyzed using cytofluorimetric strategy with the following surface markers: CD71 (PE Mouse Anti-Human CD71, BD Biosciences), glycophoryin A (APC Mouse Anti-Human CD235a, BD Biosciences). After 9 days in differentiation culture, cell viability was greater than 60% in all samples and more than 80% of cells transduced with lentivirus expressing shRNA of FECH showed an increase in PPIX, determined by flow cytometry (FIG. 10). Treatment of Biotopertin (100 nM) had no negative effects on erythroid cell surface markers and reduced PPIX accumulation by 60% (FIG. 11).

While preferred embodiments of the present application have been shown and described herein, it will be obvious to those skilled in the art that such embodiments are provided by way of example only. Numerous variations, changes, and substitutions will now occur to those skilled in the art without departing from the application. It should be understood that various alternatives to the embodiments of the application described herein may be employed in practicing the application. It is intended that the following claims define the scope of the application and that methods and structures within the scope of these claims and their equivalents be covered thereby.

### Example 9: Preparation of form A of Bitopertin

### General

Form A can be produced by digestion in solvents as e.g. methanol, ethanol, 2-propanol, isopropylacetate, t-butyl methyl ether, toluene or solvent mixtures as acetone/water (e.g. 1:1, w/w), water/methanol (e.g. 1:1, w/w), water/ethanol (e.g. 0.4:0.6, w/w). It can also be prepared by re-crystallization of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone with or without seeding in solvent systems comprising but not limited to ethanol, water/ethanol (e.g. 0.6:0.4, w/w).

Crystallization Procedure 30.0 g of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone were dissolved in 150 g ethanol and heated up to 70° C. The solution was hot filtered. The temperature was reduced to 40-42° C. At 40-42° C. 300 mg of form A seeding crystals of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone were added. The temperature was hold for 1 h at 40-42° C. Subsequently the suspension was cooled with 0.3 K/min down to 0 to −5° C. After stirring at 0 to −5° C. for 1 h the crystals were filtered, washed with ca. 20 mL of ethanol (0 to −5° C.) and dried at 50° C./0-20 mbar for 14 h. Yield: 26.31 g (87.7%).

### Preparation of Seeding Crystals of Form A

Form A seeding crystals can be prepared by digestion of a slurry of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone in solvent systems comprising but not limited to ethanol, methanol and water mixtures of ethanol/water (e.g. 0.4:0.6 w/w). After stirring the slurry at room temperature for several days form A crystals could be filtered and were dried at 50° C./0-20 mbar for 14 h. It might be necessary to repeat this procedure several times.

### Solid State Properties of Form A

XRPD-pattern, IR-spectrum, DSC curve, and TG curve of form A were measured using the methods described herein, and are shown in FIGS. 12 to 15.

XRPD patterns were recorded at ambient conditions in transmission geometry with a STOE STADI P diffractometer (Cu Ka radiation, primary monochromator, position sensitive detector, angular range 3 to 42 2Theta (deg), approximately 60 minutes total measurement time). The samples were prepared and analyzed without further processing (e.g. grinding or sieving) of the substance.

Alternatively, X-ray diffraction patterns were recorded in transmission geometry with a STOE STADIP diffractometer with CuKa radiation (1.54 A) and a position sensitive detector. The samples (approximately 50 mg) were prepared between thin polymer (or aluminum) films and analyzed without further processing (e.g. grinding or sieving) of the substance.

X-ray diffraction patterns were also measured on a Scintag X1 powder X-ray diffractometer equipped with a sealed copper Ka 1 radiation source. The samples were scanned from 2 to 36 2Theta (deg) at a rate of 1 degree 2Theta per minute with incident beam slit widths of 2 and 4 mm and diffracted beam slit widths of 0.3 and 0.2 mm.

For single crystal structure analysis a single crystal was mounted in a loop on a goniometer and measured at ambient conditions. Alternatively, the crystal was cooled in a nitrogen stream during measurement. Data were collected on a STOE Imaging Plate Diffraction System (IPDS) from STOE (Darmstadt). In this case Mo-radiation of 0.71 A wavelength was used for data collection. Data was processed with STOE IPDS-software. The crystal structure was solved and refined with standard crystallographic software. In this case the program ShelXTL from Bruker AXS (Karlsruhe) was used.

Alternatively, for synchrotron radiation was used for data collection. A single crystal was mounted in a loop and cooled to approximately 100 K in a nitrogen stream. Data was collected at the Swiss Light Source beamline XIOSA using a MAR CCD225 detector with synchrotron radiation and data processed with the program XDS. The crystal structure was solved and refined with standard crystallographic software. In this case the program ShelXTL from Bruker AXS (Karlsruhe) was used. The crystal structure was solved and refined with ShelXTL (Bruker AXS, Karlsruhe).

The IR-spectrum of a sample was recorded as film of a Nujol suspension consisting of approx. 5 mg of sample and few Nujol between two sodium chloride plates, with an FT-IR spectrometer in transmittance. The Spectrometer was a Nicolet™ 20SXB or equivalent (resolution: 2 cm^{-1, 32} or more coadded scans, MCT detector).

DSC curves were recorded using a Mettler-Toledo™ differential scanning calorimeter DSC820 or DSC 821 with a FRS05 sensor. In some embodiments, system suitability tests and calibrations were carried out according to the internal standard operation procedure. For the measurements of crystalline form A, approximately 2-6 mg of a sample was placed in aluminum pans, accurately weighed and hermetically closed with perforation lids. Prior to measurement, the lids were automatically pierced resulting in approx. 1.5 mm pin holes. The sample was then heated under a flow of nitrogen of about 100 mL/min using heating rates of 10 K/min.

The TGA curves were measured on a Mettler-Toledo™ thermogravimetric analyzer (TGA850 or TGA851). In certain embodiments, system suitability tests and calibrations were carried out according to the internal standard operation procedure.

For the thermogravimetric analyses, approx. 5 to 10 mg of sample was placed in aluminum pans, accurately weighed and hermetically closed with perforation lids. Prior to measurement, the lids were automatically pierced resulting in approx. 1.5 mm pin holes. The sample was then heated under a flow of nitrogen of about 50 mL/min using a heating rate of 5 K/min.

### Example 10: Preparation of form B of Bitopertin

### General

Form B can be prepared by re-crystallization of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone with or without seeding in different solvent systems comprising methanol, ethanol, 1,4-dioxane and water mixtures of these.

### Crystallization Procedure

30.0 g of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone were dissolved in 150 g ethanol and heated up to 60° C. Dissolution of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone occurred between 55-57° C. The solution was hot filtered. The temperature was reduced to 40-42° C. At 40-42° C. 3.0 g (10%-w) of form B seeding crystals of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone were added to the clear solution. Subsequently the suspension was cooled down to 5° C. within 5 hours. The crystals were filtered, washed with ca. 10 mL of ethanol (0° C.) and dried at 50° C./0-20 mbar for 14 h. Yield: 29.17 g (88.4%).

### Preparation of Seeding Crystals of Form B

Form B seeding crystals can be prepared by rapid cooling of a highly saturated solution of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone in solvent systems comprising but not limited to ethanol, tetrahydrofurane, toluene or 1,4-dioxane. 3.0 g of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone were dissolved in 9 g ethanol and heated up to 70° C. The solution was hot filtered. The temperature of the clear solution was rapidly reduced to 0 to −5° C. The crystals were filtered, washed with ca. 20 mL of ethanol (0 to −5° C.) and dried at 50° C./0-20 mbar for 14 h. It might be necessary to repeat this procedure several times.

### Solid State Properties of Form B

XRPD-pattern, IR-spectrum, DSC curve, and TG curve of form B were measured using the methods described herein and are listed in FIGS. 16 to 19.

### Example 11: Preparation of form C of Bitopertin

### General

Form C can be produced by digestion in solvents as n-heptane, toluene, o-xylene or solvent mixtures as n-heptane/toluene (e.g. 1:0.8, w/w). It can also be prepared by re-crystallization of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone with seeding in different solvent systems.

Crystallization Procedure 45.0 g of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone were digested in 43.4 g toluene and 54.7 g n-heptane and heated up to 98-100° C. The suspension was stirred at 98-100° C. for 48 h. The suspension was hot filtered. The obtained solid residues were dried at 70° C./0-20 mbar for 24 h. Yield: 23.0 g (51.5%).

### Solid State Properties of Form C

XRPD-pattern, IR-spectrum, DSC curve, and TG curve of form C were measured using the methods disclosed herein and are listed in FIGS. 20 to 23.

### Example 12: Preparation of Amorphous Form of Bitopertin

### General

An amorphous form was accessible from ethanol solution upon fast evaporation at approx. 40° C. under vacuum. Alternatively, amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone was accessible by lyophilization.

Preparation Procedure 0.50 g of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone were dissolved in 50 g of ethanol at 65° C. While spinning (rotary evaporator) at 40° C. maximum vacuum was applied. After complete evaporation of the solvent, the solid was further dried at ca. 25° C./5-20 mbar for 18 h. Analysis revealed amorphous [4-(3-fluoro-5- trifluoromethylpyridin-2-yl) piperazin-1-yl][5-methylsulfonyl-2-[((S)-2,2,2-trifluoro-1-methylethyl)oxy]phenyl]methanone.

### Solid State Properties of the Amorphous Form

XRPD-pattern, IR-spectrum, DSC curve, and TG curve and moisture sorption/desorption isotherms of the amorphous form were measured using the methods described herein and are listed in FIGS. 24 to 28.

### Example 13: Preparation of a Methylparaben Cocrystal Form of Bitopertin

### General

Cocrystals of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone and methylparaben can be produced by digestion in solvents as e.g. ethanol and water. It can also be prepared by recrystallization of form A, B, C or amorphous form of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone and methylparaben with or without seeding in solvent systems comprising but not limited to ethanol. The [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone to methylparaben ratio can range from 1:1 to 1:10. In some embodiments, the ratio of Bitopertin and methylparaben in a cocrystal is 1:1.

### Preparation Procedure

100 mg of [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone and 28 mg methylparaben (1 part [4-(3-fluoro-5-trifluoromethylpyridin-2-yl) piperazin-1-yl][5-methylsulfonyl-2-[((S)-2,2,2-trifluoro-1-methylethyl)oxy]phenyl]methanone+1 part methylparaben) were dissolved in 0.3 mL ethanol and heated up to dissolve both substances. The clear solution was cooled down to room temperature without stirring.

After 7 weeks the crystals were filtered, washed with ethanol/water (60/40 w/w) and dried at room temperature/0-20 mbar for 14 h.

### Solid State Properties of the Methylparaben Cocrystal Form

XRPD-pattern, IR-spectrum, DSC curve, and TG curve of the methylparaben cocrystal were measured using the methods described herein and are listed in FIGS. 29 to 32.

### INCORPORATION BY REFERENCE

All references cited in this application, and their references, are incorporated by reference herein in their entirety where appropriate for teachings of additional or alternative details, features, and/or technical background.

1. A method of treating erythropoietic protoporphyria (EPP) or X-linked protoporphyria (XLPP) in a subject comprising administering a pharmaceutical composition comprising a solid form of Bitopertin having the following structure:

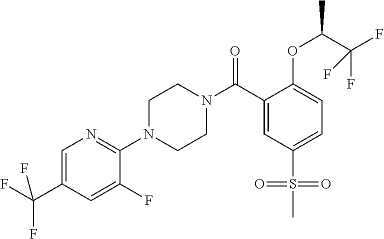 [SMILES: C[C@H](Oc1ccc(S(C)(=O)=O)cc1C(=O)N1CCN(c2ncc(C(F)(F)F)cc2F)CC1)C(F)(F)F]

2. (canceled)

3. The method of claim 1, wherein the subject has acute and/or cutaneous photosensitivity.

4-6. (canceled)

7. The method of claim 1, wherein the subject has EPP.

8. The method of claim 1, wherein the subject has XLPP.

9. (canceled)

10. The method of claim 3, wherein the acute photosensitivity is due to sun exposure.

11. The method of claim 1, wherein the method increases pain free light exposure in the subject.

12. The method of claim 1, wherein the method decreases light sensitivity in the subject.

13-16. (canceled)

17. The method of claim 1, wherein accumulation of PPIX is inhibited.

18. The method of claim 17, wherein the accumulation of PPIX is inhibited in a dose dependent manner.

19. (canceled)

20. The method of claim 1, wherein the solid form of Bitopertin demonstrates an EC50 of less than 100 nM.

21-22. (canceled)

23. The method of claim 1, wherein the subject has PPIX levels that are at least 10%, 20%, 30%, 40%, or 50% more than PPIX levels in a healthy subject prior to administration of the solid form of Bitopertin.

24-27. (canceled)

28. The method of claim 1, wherein the subject's PPIX levels decrease while the patient's heme levels are substantially maintained.

29-31. (canceled)

32. The method of claim 1, wherein the subject's heme levels decrease by not more than 10%.

33. (canceled)

34. The method of claim 1, wherein the subject's free-protoporphyrin IX levels decrease by at least 50%.

35-36. (canceled)

37. The method of claim 1, wherein the method decreases protoporphyrin IX levels in the stool of the subject.

38-43. (canceled)

44. The method of claim 1, wherein the method decreases protoporphyrin IX levels in the skin of the subject.

45-49. (canceled)

50. The method of claim 1, wherein the method decreases protoporphyrin IX levels in the skin of the subject to less than 0.5 FDU.

51-55. (canceled)

56. The method of claim 1, wherein the method decreases protoporphyrin IX levels in the erythrocytes of the subject.

57-60. (canceled)

61. The method of claim 1, wherein the method decreases protoporphyrin IX levels in the erythrocytes of the subject to levels less than 53 μmol L^{-1.}

62-68. (canceled)

69. The method of claim 1, wherein the method decreases zinc-protoporphyrin IX levels in the subject's erythrocytes.

70-88. (canceled)

89. The method of claim 1, comprising further administering to the subject an additional active agent and/or supportive therapy.

90. The method of claim 89, wherein the additional active agent and/or supportive therapy is selected from the group consisting of: avoiding sunlight, topical sunscreens, skin protection, UVB phototherapy, Afamelanotide (Scenesse®), bortezomib, proteasome inhibitors, chemical chaperones, cholestyramine, activated charcoal, iron supplementation, liver transplantation, bone marrow transplantation, splenectomy, and blood transfusion.

91. The method of claim 1, wherein the solid form of Bitopertin is selected from the group consisting of: crystalline form A, crystalline form B, crystalline form C, methylparaben cocrystal form, and an amorphous form of Bitopertin.

92. The method of claim 1, wherein the solid form of Bitopertin is a crystalline form A of Bitopertin, characterized by at least three peaks selected from the following X-ray diffraction peaks obtained with a CuKa radiation, expressed in degrees 2Theta=13.1, 14.3, 15.4, 16.2, 17.1, 17.2, 17.6, 18.0, 19.8, 20.1, 20.4, 21.0, 22.6 and 24.3 (±0.2).

93. The method of claim 92, wherein the crystalline form A is characterized by the following X-ray diffraction peaks obtained with a CuKa radiation, expressed in degrees 2Theta=13.1, 14.3, 15.4, 16.2, 17.1, 17.2, 17.6, 18.0, 19.8, 20.1, 20.4, 21.0, 22.6 and 24.3 (±0.2).

94-96. (canceled)

97. The method of claim 1, wherein the solid form of Bitopertin is a crystalline form B of Bitopertin, characterized by at least three peaks selected from the following X-ray diffraction peaks obtained with a CuKa radiation, expressed in degrees 2Theta=11.4, 15.4, 16.2, 16.4, 17.8, 18.3, 19.2, 20.1, 21.0, 22.0, 22.5, 26.4 (±0.2).

98. The method of claim 97, wherein the crystalline form B is characterized by the following X-ray diffraction peaks obtained with a CuKa radiation, expressed in degrees 2Theta=11.4, 15.4, 16.2, 16.4, 17.8, 18.3, 19.2, 20.1, 21.0, 22.0, 22.5, 26.4 (±0.2).

99-101. (canceled)

102. The method of claim 1, wherein the solid form of Bitopertin is a crystalline form C of Bitopertin, characterized by at least three peaks selected from the following X-ray diffraction peaks obtained with a CuKa radiation, expressed in degrees 2Theta=14.9, 15.7, 16.7, 17.7, 17.8, 18.7, 19.7, 21.8, 22.0 and 25.2 (±0.2).

103. The method of claim 102, wherein the crystalline form C is characterized by the following X-ray diffraction peaks obtained with a CuKa radiation, expressed in degrees 2Theta=14.9, 15.7, 16.7, 17.7, 17.8, 18.7, 19.7, 21.8, 22.0 and 25.2 (±0.2).

104-106. (canceled)

107. The method of claim 1, wherein the solid form of Bitopertin is an amorphous form of Bitopertin, characterized by an absence of sharp X-ray peaks in its XRPD pattern, and/or by an infrared spectrum having sharp bands at 1642, 1622, 1599, 1579, 1509, 1487, 1399, 1329, 1293, 1253, 1159, 1124, 1090, 1016, 960, 920, 903, 889, 827, 782, 763, 739 and 636 cm−1 (±3 cm−1).

108. (canceled)

109. The method of claim 1, wherein the solid form of Bitopertin is a methylparaben cocrystal form, characterized by at least three peaks selected from the following X-ray diffraction peaks obtained with a CuKa radiation, expressed in degrees 2Theta=8.0, 8.9, 10.5, 12.6, 15.2, 16.1, 17.7, 18.5, 19.8, 20.2, 21.7, 22.9, 24.2 and 25.9 (±0.2).

110. The method of claim 109, wherein the methylparaben cocrystal form is characterized by the following X-ray diffraction peaks obtained with a CuKa radiation, expressed in degrees 2Theta=8.0, 8.9, 10.5, 12.6, 15.2, 16.1, 17.7, 18.5, 19.8, 20.2, 21.7, 22.9, 24.2 and 25.9 (±0.2).

111-113. (canceled)

114. The method of claim 1, wherein the pharmaceutical composition further comprises a pharmaceutically acceptable carrier.

115. (canceled)


SEQUENCE LISTING (5 sequences)

SEQ ID NO: 1 - DNA, 20 bp, Artificial Sequence
  Description of Artificial Sequence Synthetic oligonucleotide
atgggaggccctgaaactct

SEQ ID NO: 2 - DNA, 20 bp, Artificial Sequence
  Description of Artificial Sequence Synthetic oligonucleotide
tgaaactcttggagatgttc

SEQ ID NO: 3 - DNA, 20 bp, Artificial Sequence
  Description of Artificial Sequence Synthetic oligonucleotide
tctgagactcttcttggacc

SEQ ID NO: 4 - DNA, 58 bp, Artificial Sequence
  Description of Artificial Sequence Synthetic oligonucleotide
ccgggctttgcagatcatattctaactcgagttagaatatgatctgcaaagctttttg

SEQ ID NO: 5 - DNA, 58 bp, Artificial Sequence
  Description of Artificial Sequence Synthetic oligonucleotide
aattcaaaaagctttgcagatcatattctaactcgagttagaatatgatctgcaaagc